SEQUENCE LISTING (8 sequences)

SEQ ID NO: 1 - DNA, 639 bp, hepatitis B virus
  CDS (1)..(639): hepatitis B virus ayw subtype core antigen (HBcAg)
atgcaactttttcacctctgcctaatcatctcttgttcatgtcctactgttcaagcctccaagctgtgccttgggtggctttggggcatggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 2 - protein, 212 aa, hepatitis B virus
  hepatitis B virus ayw subtype core antigen (HBcAg)
MQLFHLCLIISCSCPTVQASKLCLGWLWGMDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 3 - protein, 97 aa, influenza A virus
  influenza A virus strain A/34/PR8 full length M2 protein
MSLLTEVETPIRNEWGCRCNGSSDPLTIAANIIGILHLTLWILDRLFFKCIYRRFKYGLKGGPSTEGVPKSMREEYRKEQQSAVDADDGHFVSIELE

SEQ ID NO: 4 - DNA, 1860 bp, Artificial Sequence
  CoHo7sAg,eGFP nucleotide sequence
atggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtaataatctggaaggatcccctctaattccaggatcatcaacaaccagcaccggaccatgcaaaacctgcacaactcctgctcaaggaacctctatgtttccctcatgttgctgtacaaaacctacggacggaaattgcacctgtattcccatcccatcatctagcggccgcgatccggcgagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattccgccggcgtatcgtccgccgaacgcgccaattctgagcacgctgccggagaccaccgtggttggtgggagctctggtggcagcggcggcagcggtggtagcggtggcagcggtggtagcggcgggtcgactatggatatcgacccatataaagaatttggcgcgacggttgagctgctgagctttctgccaagcgatttctttccgagcgtccgcgacctgctggataccgccagcgcactgtatcgtgaagccctggagagcccggaacattgcagcccgcatcatacggccctgcgtcaggcaatcctgtgctggggcgaactgatgaccctggcaacctgggtcggcaataatctggaattcggcggtagcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccgggggcgctagcgacccagcaagccgtgatctggttgttaattacgtgaacaccaacatgggcctgaagatccgccaactgctgtggtttcatatcagctgtctgacgtttggccgcgagacggtgctggaatacctggttagctttggcgtttggattcgtacgccaccggcctaccgcccaccaaacgcaccgattctgagcacgctgccggaaacgacggttgtt

SEQ ID NO: 5 - protein, 620 aa, Artificial Sequence
  CoHo7sAg,eGFP amino acid sequence
MDIDPYKEFGATVELLSFLPSDFFPSVRDLLDTASALYREALESPEHCSPHHTALRQAILCWGELMTLATWVGNNLEGSPLIPGSSTTSTGPCKTCTTPAQGTSMFPSCCCTKPTDGNCTCIPIPSSSGRDPASRDLVVNYVNTNMGLKIRQLLWFHISCLTFGRETVLEYLVSFGVWIPPAYRPPNAPILSTLPETTVVGGSSGGSGGSGGSGGSGGSGGSTMDIDPYKEFGATVELLSFLPSDFFPSVRDLLDTASALYREALESPEHCSPHHTALRQAILCWGELMTLATWVGNNLEFGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGASDPASRDLVVNYVNTNMGLKIRQLLWFHISCLTFGRETVLEYLVSFGVWIRTPPAYRPPNAPILSTLPETTVV

SEQ ID NO: 6 - DNA, 1197 bp, Artificial Sequence
  CoHo7sAg,eM2e nucleotide sequence
atggacatcgatccgtacaaagaatttggcgcgaccgtcgagctgctgagcttcctgccgagcgattttttcccgagcgtgcgtgacctgctggacaccgcgagcgcactgtatcgtgaagcactggaaagcccagagcactgtagcccgcaccacaccgccctgcgccaggcgattctgtgctggggtgaactgatgaccctggccacctgggtgggtaataatctggaaggatcccctctaattccaggatcatcaacaaccagcaccggaccatgcaaaacctgcacaactcctgctcaaggaacctctatgtttccctcatgttgctgtacaaaacctacggacggaaattgcacctgtattcccatcccatcatctagcggccgcgatccggcgagccgtgatctggtcgtcaactatgtgaataccaacatgggtctgaaaattcgtcagctgctgtggtttcatattagctgcctgaccttcggtcgtgaaaccgtgctggagtatctggtgagcttcggtgtgtggattccgccggcgtatcgtccgccgaacgcgccaattctgagcacgctgccggagaccaccgtggttggtgggagctctggtggcagcggcggcagcggtggtagcggtggcagcggtggtagcggcgggtcgactatggatatcgacccatataaagaatttggcgcgacggttgagctgctgagctttctgccaagcgatttctttccgagcgtccgcgacctgctggataccgccagcgcactgtatcgtgaagccctggagagcccggaacattgcagcccgcatcatacggccctgcgtcaggcaatcctgtgctggggcgaactgatgaccctggcaacctgggtcggcaataatctggaattcatgagtcttctaaccgaggtcgaaacgcctatcagaaacgaatgggggtgcagatgcaacggttcaagtgatgctagcgacccagcaagccgtgatctggttgttaattacgtgaacaccaacatgggcctgaagatccgccaactgctgtggtttcatatcagctgtctgacgtttggccgcgagacggtgctggaatacctggttagctttggcgtttggattcgtacgccaccggcctaccgcccaccaaacgcaccgattctgagcacgctgccggaaacgacggttgtt

SEQ ID NO: 7 - protein, 399 aa, Artificial Sequence
  CoHo7sAg,eM2e amino acid sequence
MDIDPYKEFGATVELLSFLPSDFFPSVRDLLDTASALYREALESPEHCSPHHTALRQAILCWGELMTLATWVGNNLEGSPLIPGSSTTSTGPCKTCTTPAQGTSMFPSCCCTKPTDGNCTCIPIPSSSGRDPASRDLVVNYVNTNMGLKIRQLLWFHISCLTFGRETVLEYLVSFGVWIPPAYRPPNAPILSTLPETTVVGGSSGGSGGSGGSGGSGGSGGSTMDIDPYKEFGATVELLSFLPSDFFPSVRDLLDTASALYREALESPEHCSPHHTALRQAILCWGELMTLATWVGNNLEFMSLLTEVETPIRNEWGCRCNGSSDASDPASRDLVVNYVNTNMGLKIRQLLWFHISCLTFGRETVLEYLVSFGVWIRTPPAYRPPNAPILSTLPETTVV

SEQ ID NO: 8 - protein, 24 aa, influenza A virus
  influenza A virus strain A/34/PR8 M2 peptide, amino acids 1-24 of M2 protein
MSLLTEVETPIRNEWGCRCNGSSD

(19) United States
(12) Patent Application Publication
(10) Pub. No.: US 2013/0171182 A1
(43) Pub. Date: Jul. 4, 2013

(54) INFLUENZA VACCINE

(71) Applicants: Michael Anthony Whelan, London (GB); William Malcolm Rosenberg, London (GB); Lucy Phillipa Beales, London (GB)

(72) Inventors: Michael Anthony Whelan, London (GB); William Malcolm Rosenberg, London (GB); Lucy Phillipa Beales, London (GB)

(73) Assignee: iQUR Limited, London (GB)

(21) Appl. No.: 13/503,317

(22) PCT Filed: Oct. 22, 2010
(86) PCT No.: PCT/GB2010/001964

§ 371 Date: Aug. 6, 2012

(30) Foreign Application Priority Data
Oct. 23, 2009 (GB) 0918679.2

(51) Int. Cl. C07K 14/11; C07K 14/02

(52) U.S. Cl. CPC C07K 14/11; C07K 14/02
USPC 424/189.1; 530/350; 536/23.72; 514/44 R; 435/320.1; 435/69.3; 435/252.33; 435/254.2; 435/325; 435/348

(57) ABSTRACT

The invention provides a protein comprising a first and a second copy of hepatitis B core antigen (HBcAg) in tandem, in which one or both of the copies of HBcAg comprises influenza virus A surface polypeptide M2 or a fragment thereof in the e1 loop.

### FIELD O F THE INVENTION

The invention relates to fusion proteins comprising influenza virus A surface polypeptide M2 or a fragment thereof, nucleic acid molecules encoding the proteins, processes for producing the proteins, pharmaceutical compositions containing the proteins and use of the proteins in vaccination.

### BACKGROUND OF THE INVENTION

Influenza virus is a member of the Orthomyxoviridae family. There are three subtypes of influenza viruses designated A, B, and C that infect humans. The influenza virion contains a segmented negative-sense RNA genome. The enveloped influenza A virions have three membrane proteins, hemagglutinin (HA), neuraminidase (NA) and proton ion-channel protein (M2); a matrix protein (M1) just below the lipid bilayer; a ribonucleoprotein core consisting of 8 viral RNA segments and three proteins (polymerase acidic protein (PA), polymerase basic protein 1 (PB1) and polymerase basic protein 2 (PB2)); and nonstructural protein 2 (NS2). Influenza B virions have four proteins in the envelope: HA, NA, NB, and BM2. Like the M2 protein of influenza A virus, the BM2 protein is a proton channel that is essential for the uncoating process. The NB protein is believed to be an ion channel, but it is not required for viral replication in cell culture.

Influenza C viruses are somewhat different. Like the influenza A and B viruses, the core of influenza C viruses consists of a ribonucleoprotein made up of viral RNA and four proteins. The M1 protein lies just below the membrane, as in influenza A and B virions. A minor viral envelope protein is CM2, which functions as an ion channel. The major influenza C virus envelope glycoprotein is called HEF (hemagglutinin-esterase-fusion) because it has the functions of both the HA and the NA.

The HA and NA proteins are envelope glycoproteins, responsible for virus attachment and penetration of the viral particles into the cell, and are immunodominant epitopes for virus neutralization and protective immunity. However, these proteins can, and often do, change from strain to strain. Due to the variability of these two proteins, a broad spectrum, long lasting influenza vaccine has so far not been developed. The influenza vaccine commonly used has to be adapted almost every year to follow the antigenic drift of the virus. When more drastic changes occur in the virus, known as an antigenic shift, the vaccine is no longer protective.

### SUMMARY OF THE INVENTION

The invention is concerned with a highly flexible influenza virus vaccine delivery system based on tandem hepatitis B (HBV) core proteins. The inventors have demonstrated that by using tandem HBV core constructs it is possible to deliver more than one heterologous epitope simultaneously into an individual and to generate a different immune response to each heterologous epitope and to the HBV core. In particular, the inventors have demonstrated that delivery of an influenza virus M2 protein fragment as an insert in a first HBV core protein in tandem with another heterologous epitope as an insert in a second HBV core protein results in the generation of immune responses to M2, the other heterologous epitope and HBV core protein. Furthermore, vaccination of mice with this protein construct before challenge with a lethal dose of influenza virus reduced the weight loss of the mice and increased their survival.

The invention provides a protein comprising a first and a second copy of hepatitis B core antigen (HBcAg) in tandem, in which one or both of the copies of HBcAg comprises influenza virus A surface polypeptide M2 or a fragment thereof in the e1 loop.

The flexibility of the technology means that one of the copies of HBcAg may comprise a second heterologous epitope that is different from M2 or a fragment thereof in the e1 loop. The second heterologous epitope may also be derived from influenza virus. However, it may also be derived from a different pathogen or allergen.

Therefore the protein of the invention is useful for inducing an immune response to influenza virus and, depending on what other heterologous epitope(s) are present, it may also be useful for simultaneously inducing an immune response to a different pathogen or allergen.

One of the advantages of the invention is that it allows precise control over the ratio of different epitopes to be delivered in a vaccine. For example, the ratio of an epitope in a first copy of HBcAg to an epitope in a second copy of HBcAg can be precisely 1:1.

The invention also provides:

a particle comprising multiple copies of a protein of the invention;

a nucleic acid molecule encoding a protein of the invention;

a host cell comprising a nucleic acid molecule of the invention;

a process for producing a protein of the invention, which process comprises culturing a host cell containing a nucleic acid molecule which encodes the protein under conditions in which the protein is expressed, and recovering the protein;

a pharmaceutical composition comprising a protein of the invention, a particle of the invention or a nucleic acid molecule of the invention and a pharmaceutically acceptable carrier or diluent;

a protein of the invention, a particle of the invention or a nucleic acid molecule of the invention for use in a method of vaccination of the human or animal body;

use of a protein of the invention, a particle of the invention or a nucleic acid molecule of the invention for the manufacture of a medicament for vaccination of the human or animal body; and

a method of inducing an immune response in a subject, which method comprises administering to the subject a protein of the invention, a particle of the invention or a nucleic acid molecule of the invention.

### BRIEF DESCRIPTION OF THE FIGURES

FIG. 1: CoHo7sAG,eGFP nucleotide and amino acid sequences and the positions of the HBsAg and eGFP inserts.

FIG. 2: CoHo7sAg,eM2e nucleotide and amino acid sequences and the positions of the HBsAg and M2e inserts.

FIG. 3: Construct maps for (A) CoHo7sAG,eGFP and (B) CoHo7sAg,eM2e. (A) CoHo7sAg,eGFP is a homo-7-linker tandem with HBV surface antigen (residues 108-155, adw) in 1st core (e1 loop) and green fluorescent protein in the 2nd core (e1 loop). (B) CoHo7sAg,M2e is a homo-7-linker tandem with HBV surface antigen (residues 108-155, adw) in 1st core (e1 loop) and influenza M2 protein (residues 1-24) in the 2nd core (e1 loop).

FIG. 4: (A) X-ray crystallographic data shows the natural dimer formed by HBV core protein. This is the main building block of the resulting virus like particle. (B) Molecular modelling suggests that the folding of tandem-core constructs appears to be indistinguishable from monomeric cores. Note the addition of a linker sequence between the two cores ensuring that they are expressed as a single protein.

FIG. 5: (A) Electron microscopy of VLPs formed by monomeric (left) and tandem (right) core proteins. VLPs appear to have an identical morphology. (B) Cryo-electron microscopy confirms that monomeric (left) and tandem (right) core constructs fold in an identical manner.

FIG. 6: SDS-PAGE confirming expression of sAg-GFP in E-coli. The protein of interest is found at 69 kDa and is indicated by an arrow. M=Marker (Precision Plus protein standards, Bio-Rad); 1=Pooled fractions sucrose cushion supernatant; and 2=Pooled fractions sucrose cushion pellet (0.855 mg/ml).

FIG. 7: SDS-PAGE with coomassie stain (left) and anti-core western blot (right) confirming expression of sAg-M2 in E-coli. The protein of interest is found at 44 kDa and is indicated by an arrow. M=Marker (Precision Plus protein standards, Bio-Rad); 1=Pooled and dialysed S-500HR fraction #5 (˜2 μg total protein); 2=Pooled and dialysed S-500HR fraction #5 (˜1 μg total protein); 3=Pooled and dialysed S-500HR fraction #5 (˜0.5 μg total protein).

FIG. 8: (A) Anti-core responses are detected after vaccination with sAg-empty tandem core and sAg-GFP tandem core but not with empty-GFP tandem core. Engerix B and alum controls are negative. (B) Anti-GFP responses are found in both single and dual insert tandem cores (empty-GFP and sAg-GFP). (C) Engerix B immunised mice show a strong anti-sAg response. A small but detectable response is seen after immunisation with sAg-empty tandem core but is negative for sAg-GFP. All ELISAs were carried out on day +21 sera.

FIG. 9: (A) Anti core response to sAg-M2 is found regardless of the presence of adjuvant (alum). (B) A strong sAg antibody response is detected after Engerix B administration and also to sAg-M2 tandem core vaccination. Again, this appears to be independent of adjuvant. (C) The M2 response is found after vaccination with sAg-M2, however, the presence of alum clearly enhances the response. All ELISAs were carried out on day +21 sera.

FIG. 10: (A) Average weight loss was calculated for each group of mice and the percentage change shown in the graph. In mice vaccinated with sAg-M2 presented on alum, there was a noticeable decrease in the rate of weight loss. (B) Total weight loss for all animals challenged with influenza is shown. Mice vaccinated with sAg-M2 presented on alum showed an initial loss of weight that was then subsequently recovered. This was not found in either alum or Engerix B control groups.

FIG. 11: The sAg-M2 tandem core construct, presented on alum, confers a clear survival advantage after influenza challenge (75%). A smaller, but detectable, level of protection was also detected when the tandem core construct was given in the absence of adjuvant.

### BRIEF DESCRIPTION OF THE SEQUENCES

SEQ ID NO: 1 is the 183 amino acid protein of the ayw subtype plus a 29 amino acid pre-sequence of HBcAg and the corresponding nucleotide sequence.

SEQ ID NO: 2 is the 183 amino acid protein of the ayw subtype plus a 29 amino acid pre-sequence of HBcAg.

SEQ ID NO: 3 is the M2 amino acid sequence from influenza virus A strain A/34/PR8.

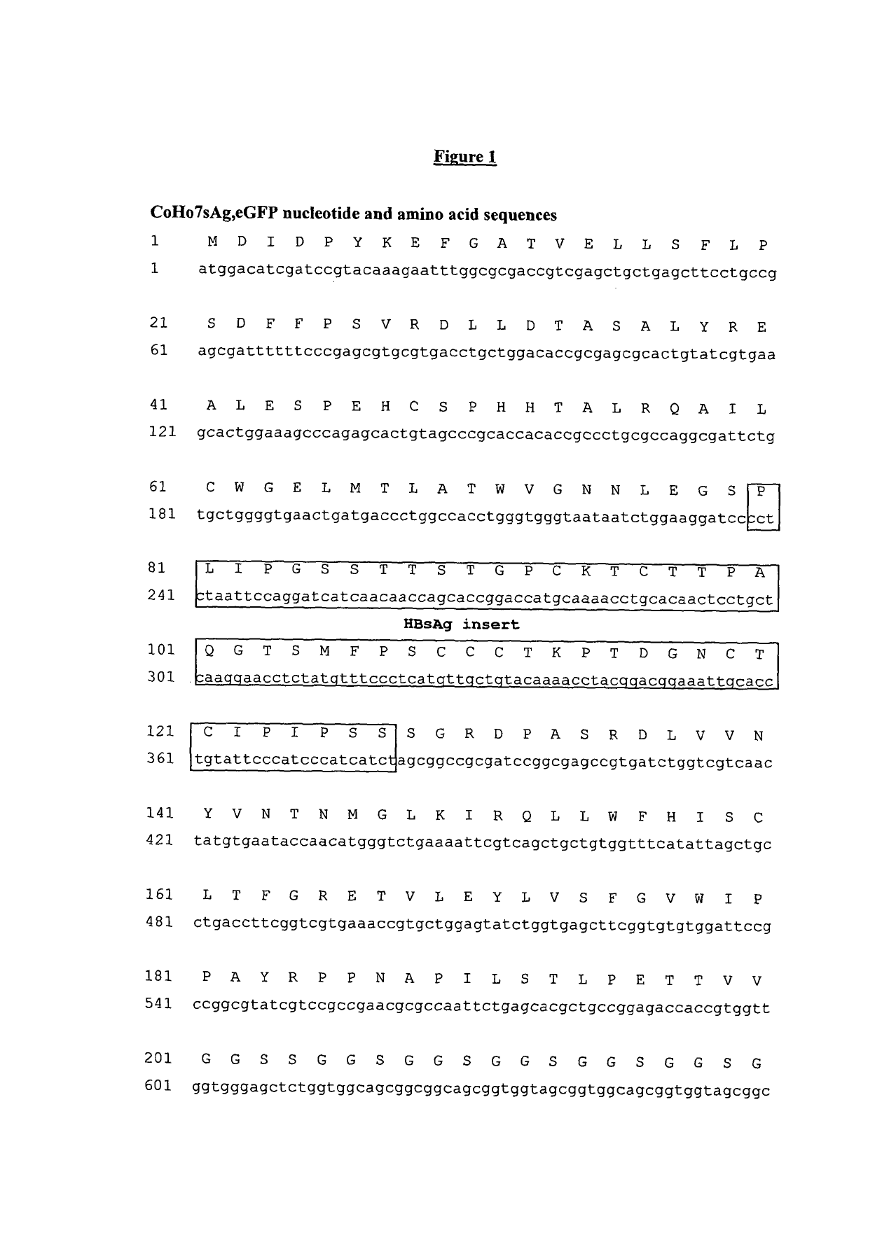

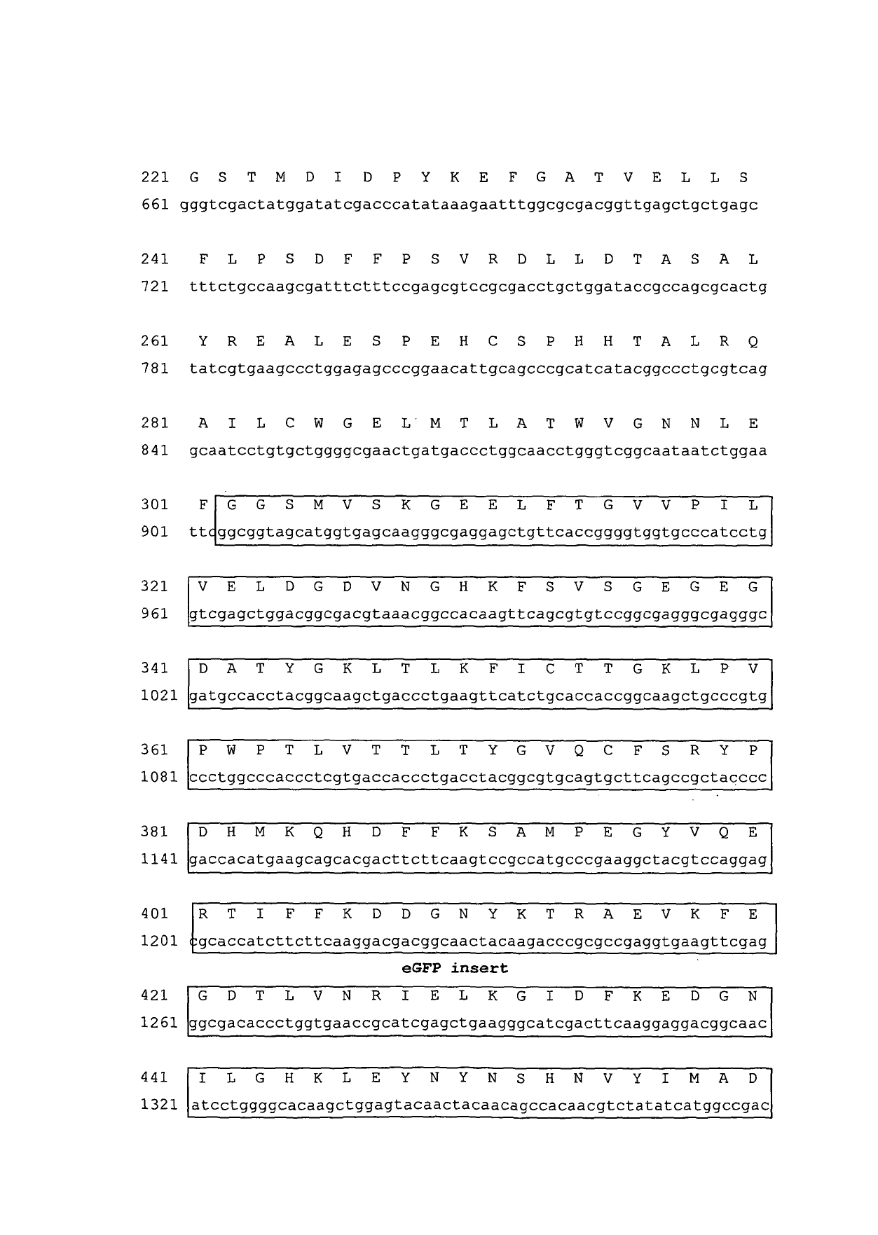

SEQ ID NO: 4 is the nucleotide and amino acid sequence of CoHo7sAg,eGFP (FIG. 1)

SEQ ID NO: 5 is the amino acid sequence of CoHo7sAg,eGFP (FIG. 1)

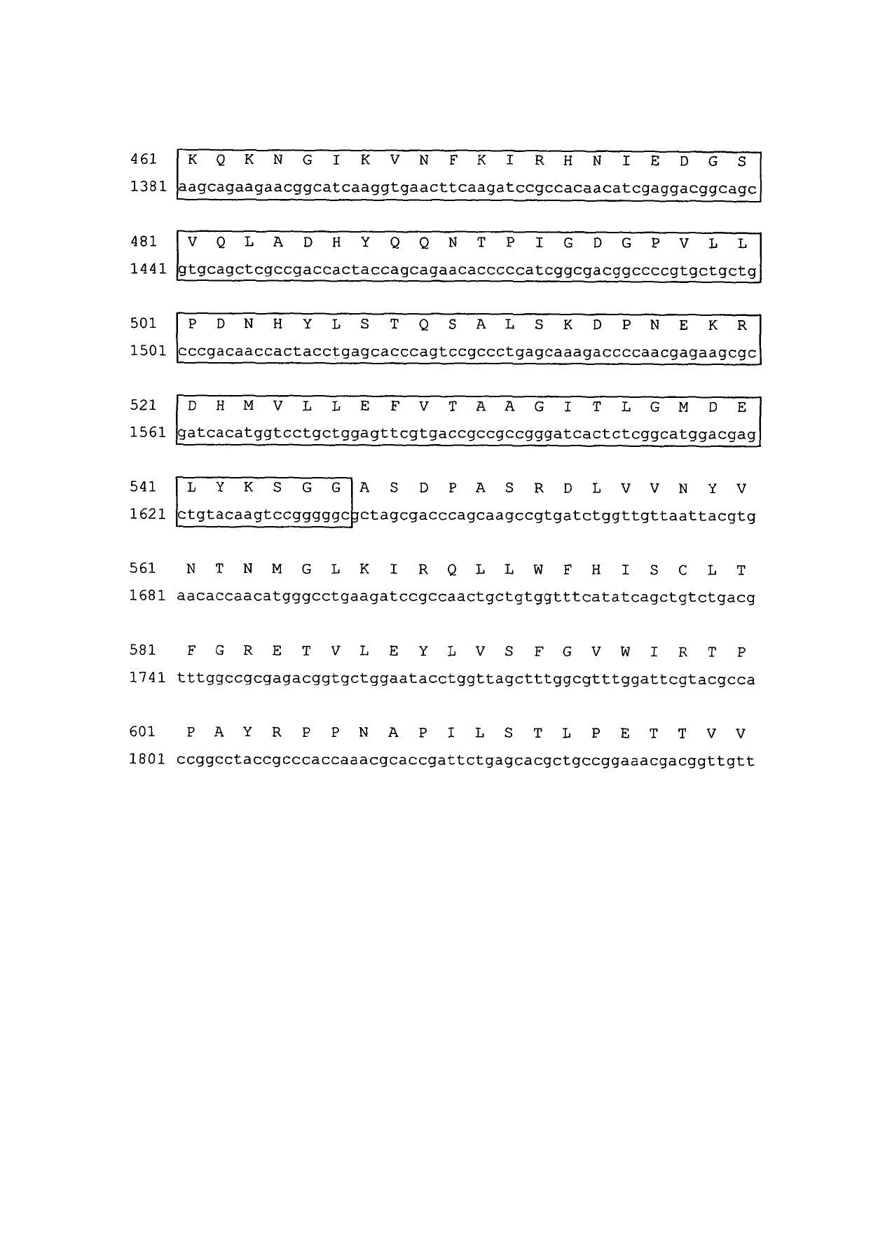

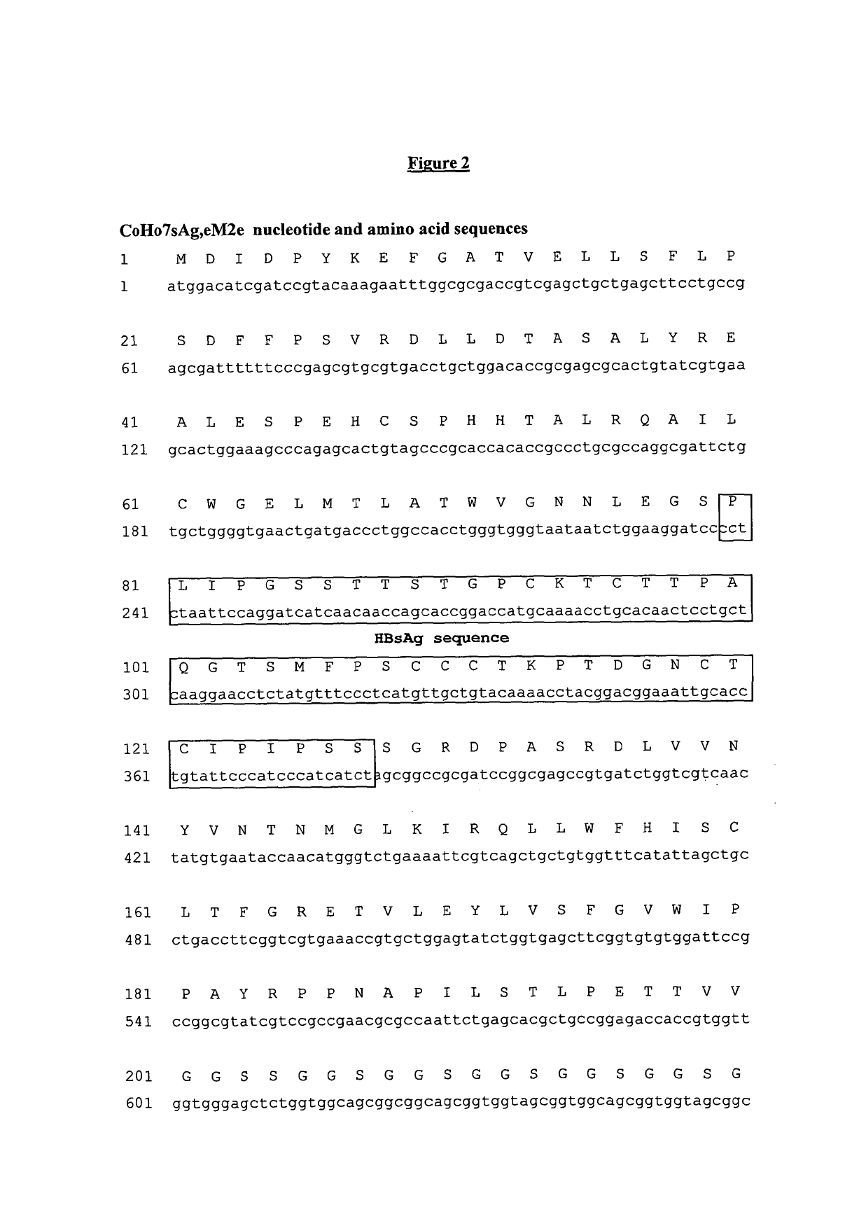

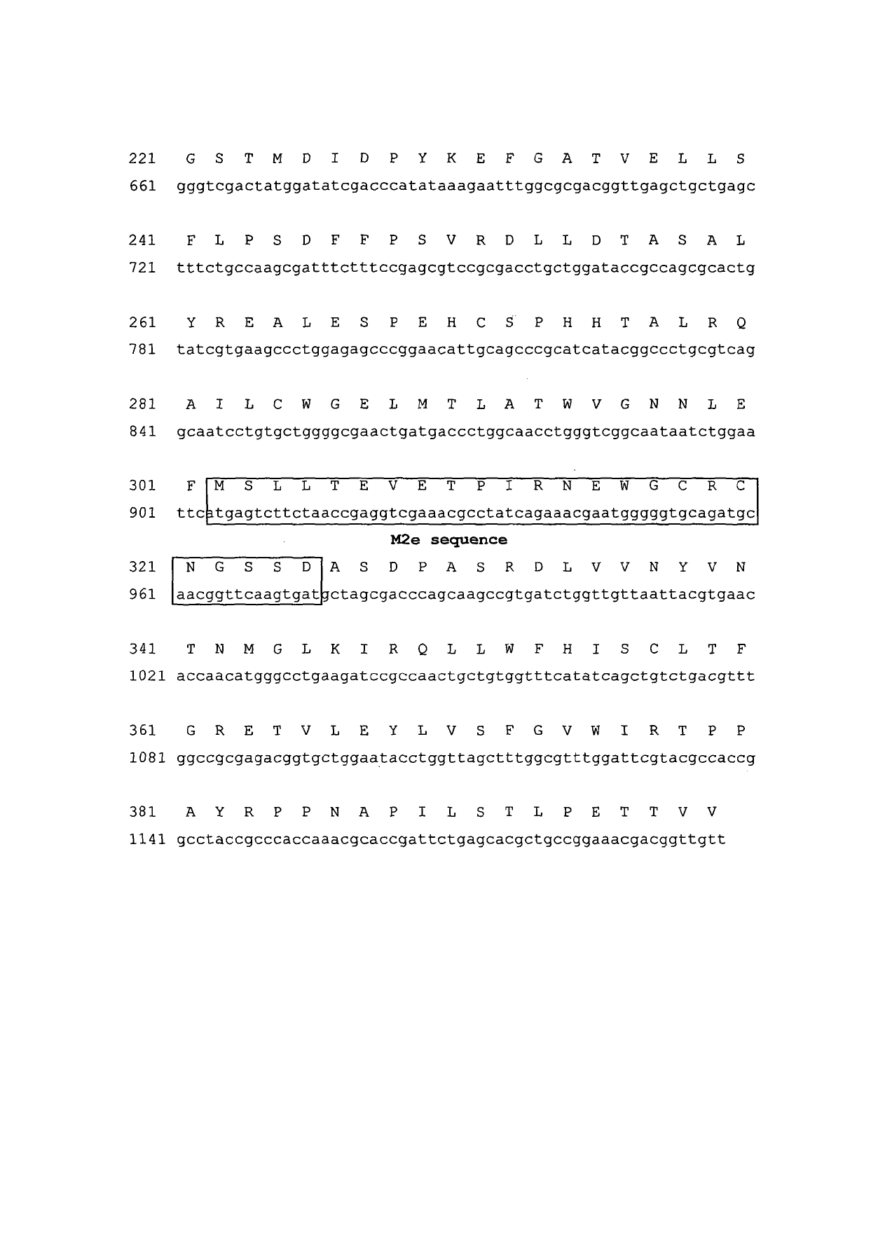

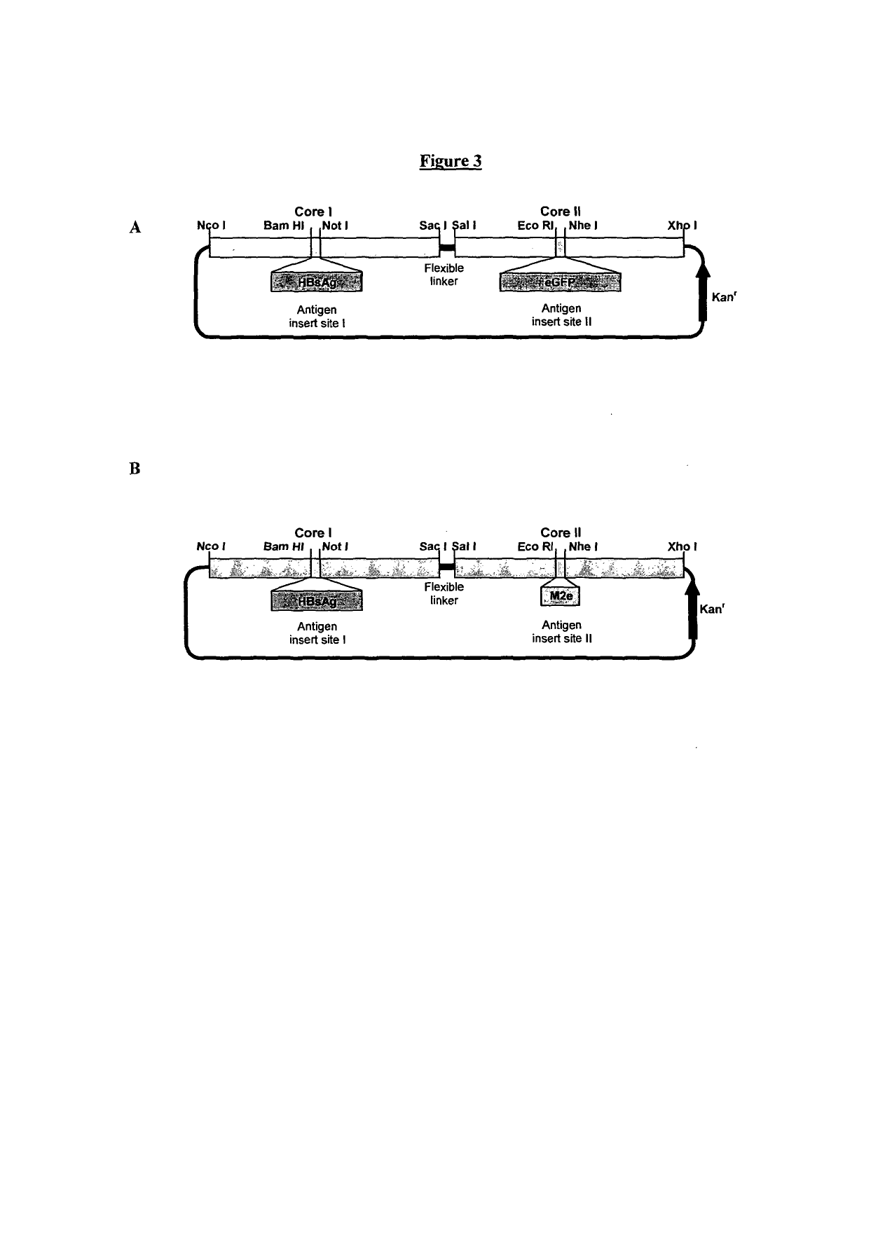

SEQ ID NO: 6 is the nucleotide and amino acid sequence of CoHo7sAg,eM2e (FIG. 2)

SEQ ID NO: 7 is the amino acid sequence of CoHo7sAg,eM2e (FIG. 2)

### DETAILED DESCRIPTION OF THE INVENTION

The highly immunogenic wild-type HBV core protein has been shown to carry a Major Insertion Region (MIR) at the tip of its spike region. Antigenic sequences from third party proteins can be inserted into these MIRs thus conferring immunogenicity onto both insert and core sequence. However, it has been shown that large or hydrophobic regions cannot be accommodated into these areas because this leads to a failure of virus-like particle (VLP) formation (Pumpens & Grens 2001).

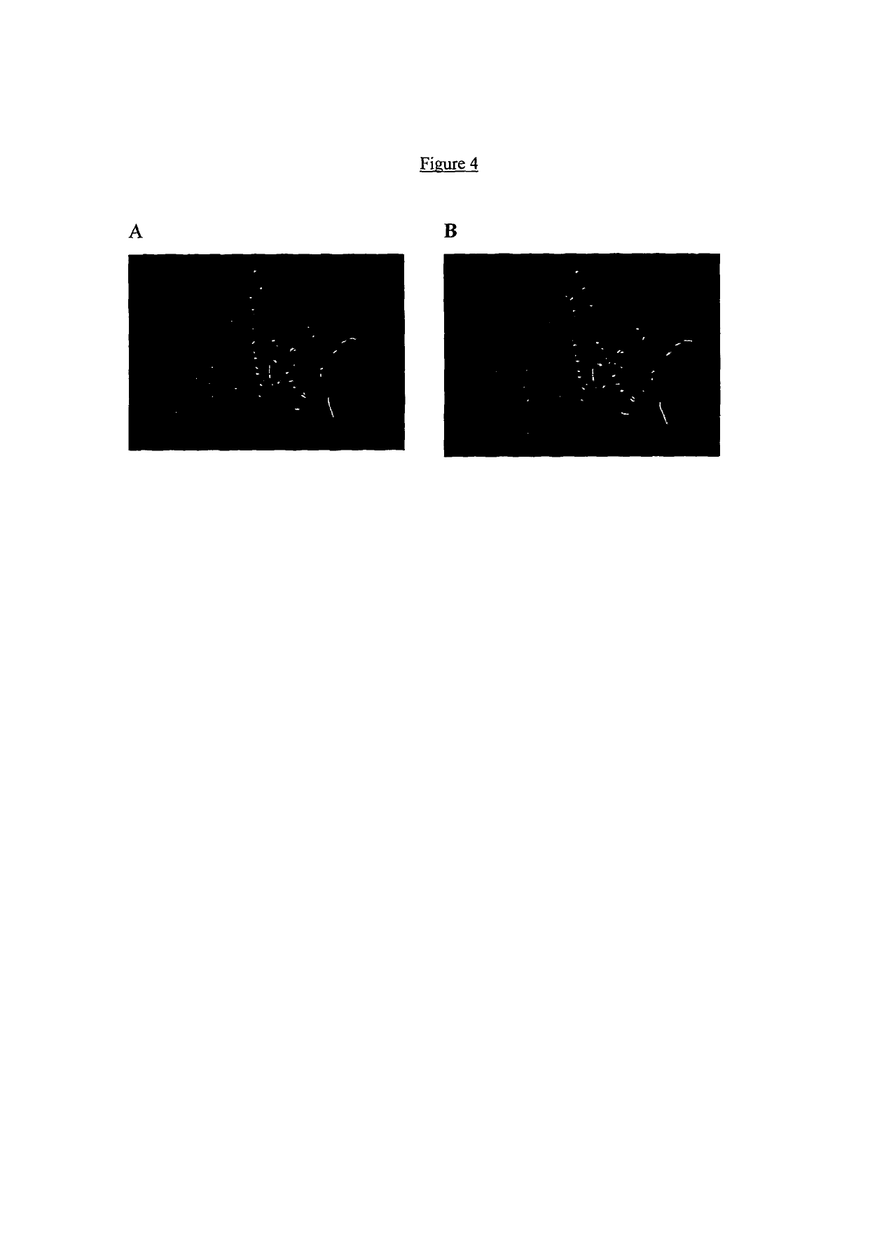

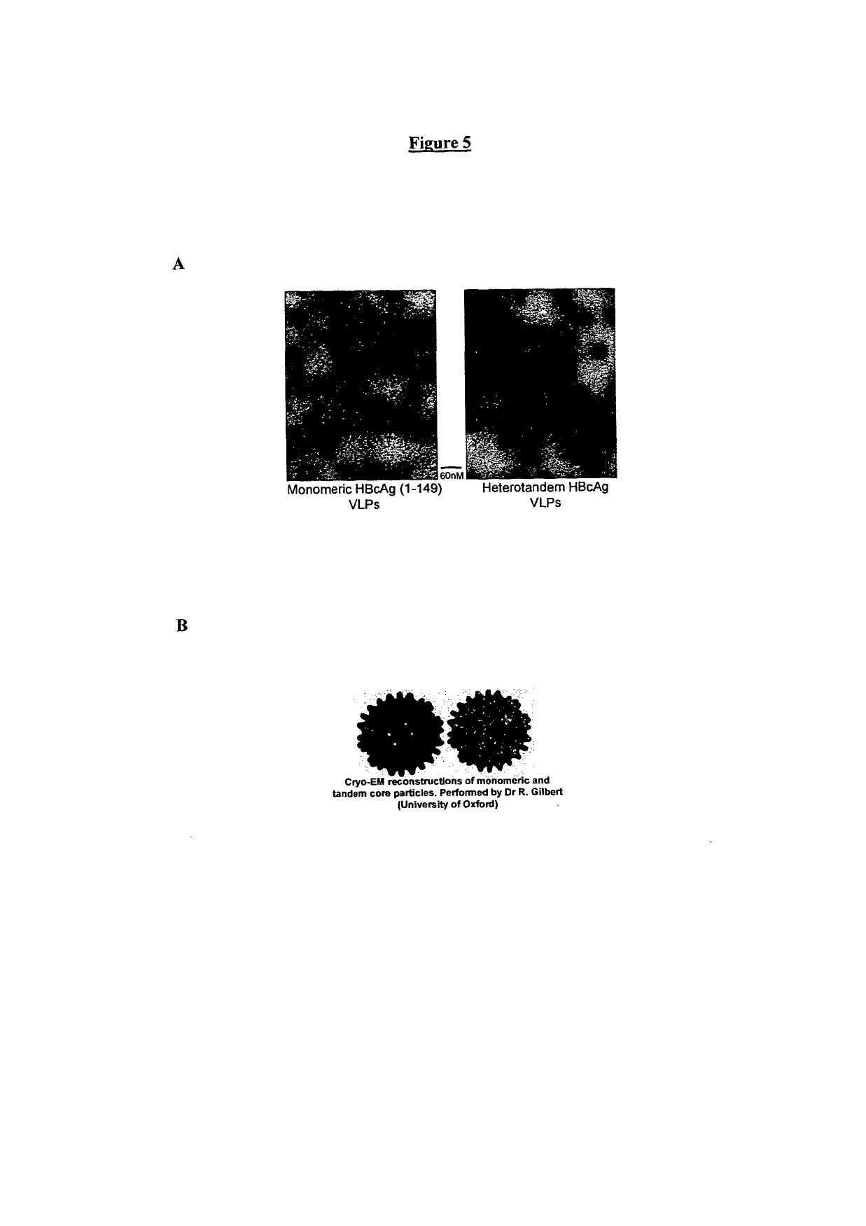

The current inventors have now established that tandem core constructs can overcome these limitations. Tandem core constructs are a genetic fusion of two hepatitis B (HBV) core genes such that the resulting recombinant protein forms two parallel “spikes” which are indistinguishable from wild type core proteins which naturally dimerise (FIG. 4). Tandem core proteins form virus like particles (VLP) in a manner similar to monomeric core protein (FIG. 5).

The inventors have now demonstrated that tandem core constructs allow two simultaneous insertions to be carried out which, when used in vivo, give rise to three simultaneous immune responses to all the components (the two inserts and the HBcAg). They have also now established that large and/or hydrophobic insertions can be accommodated without VLP disruption.

In more detail, the inventors designed and generated two different tandem core constructs, both with dual inserts (see Example for more details). The first contained HBV surface antigen (sAg) in core 1 and green fluorescent protein (GFP) in core 2 (CoHo7sAg,eGFPs construct). The second comprised sAg in core 1 but had the M2 protein from influenza in core 2 (CoHo7sAg,M2e construct). These were expressed in bacteria and the proteins (sAg-GFP and sAg-M2) were purified and then tested in vivo for immunogenicity.

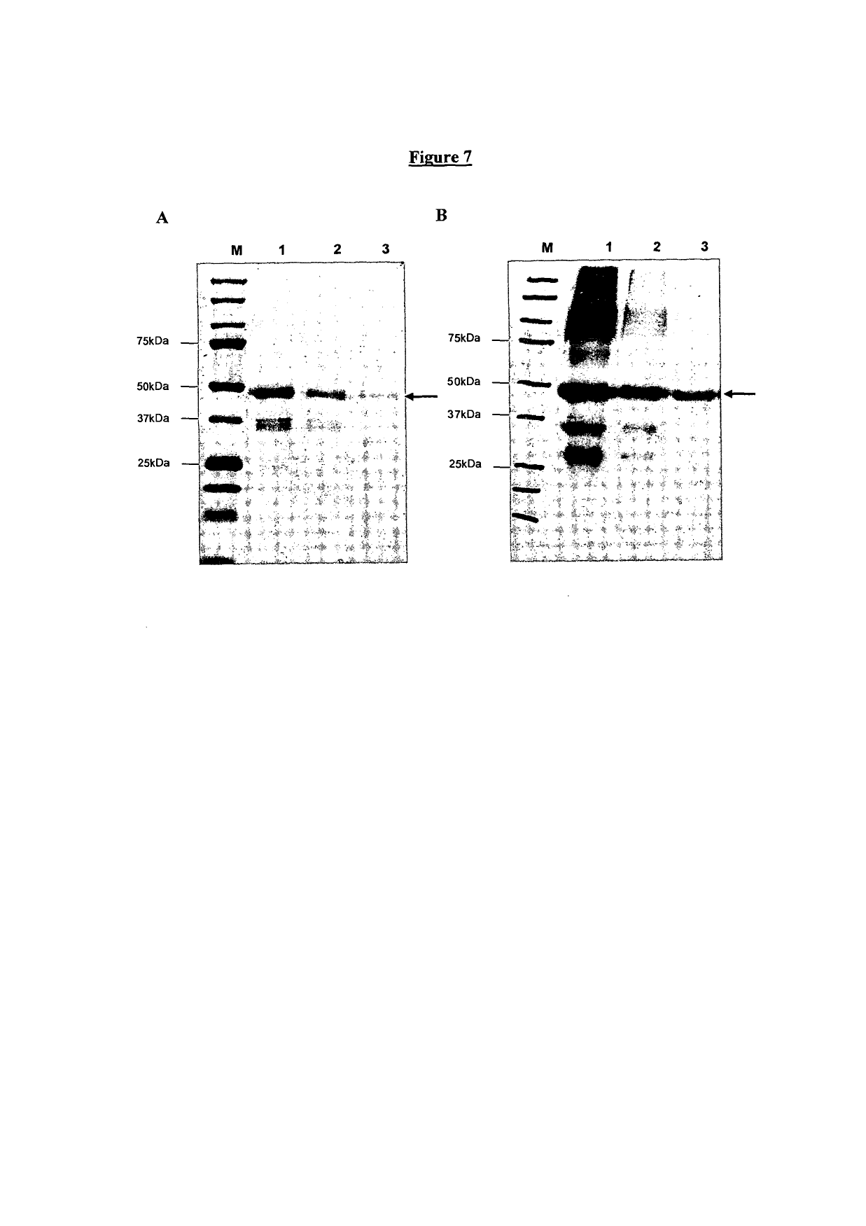

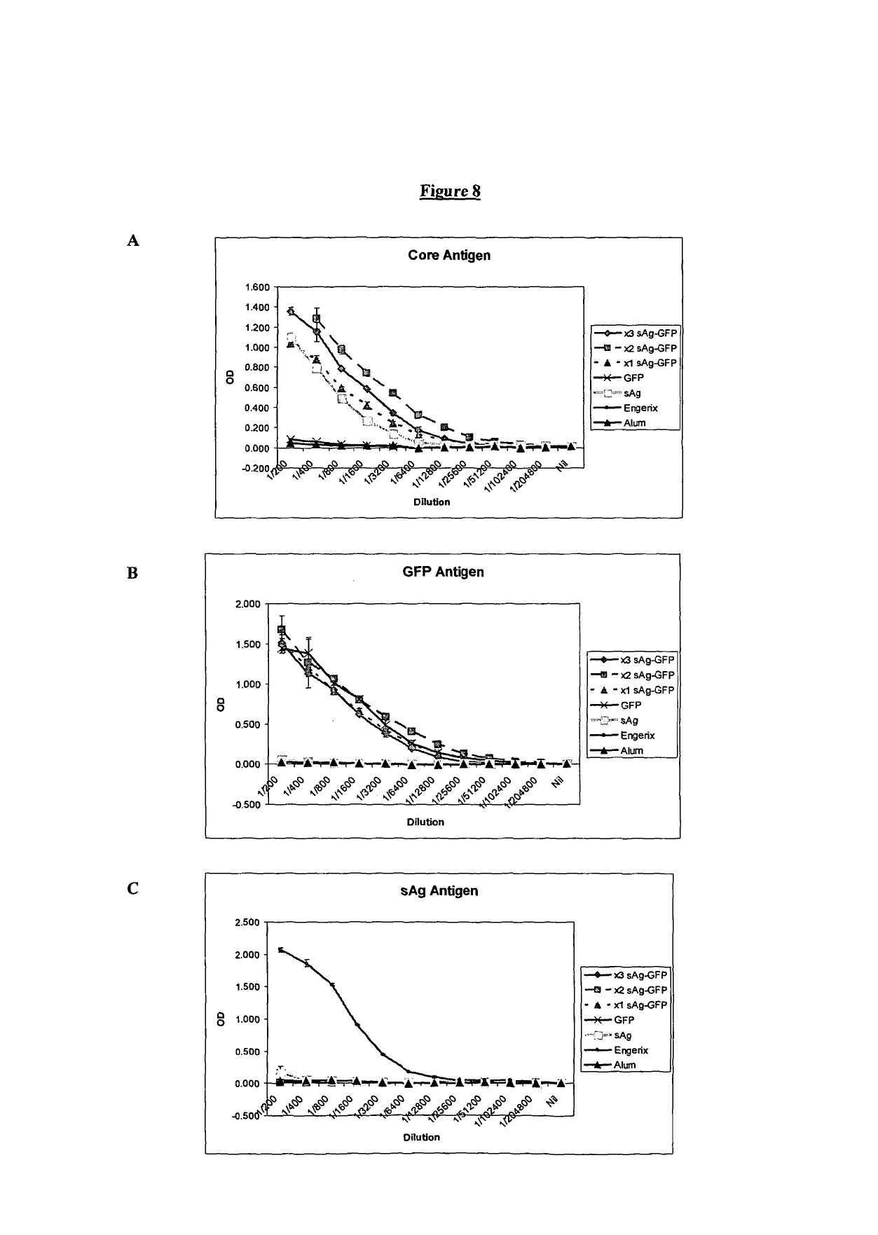

For sAg-GFP, strong immune responses were seen to GFP, but not to sAg (FIG. 8). The most likely explanation for this is that the large GFP (27 kDa) insert sterically hinders the availability of the much smaller sAg (4.8 kDa) insert. Thus, although the data clearly demonstrate that large inserts can indeed be accommodated by tandem core, care must be taken as to the orientation of the antigenic insert.

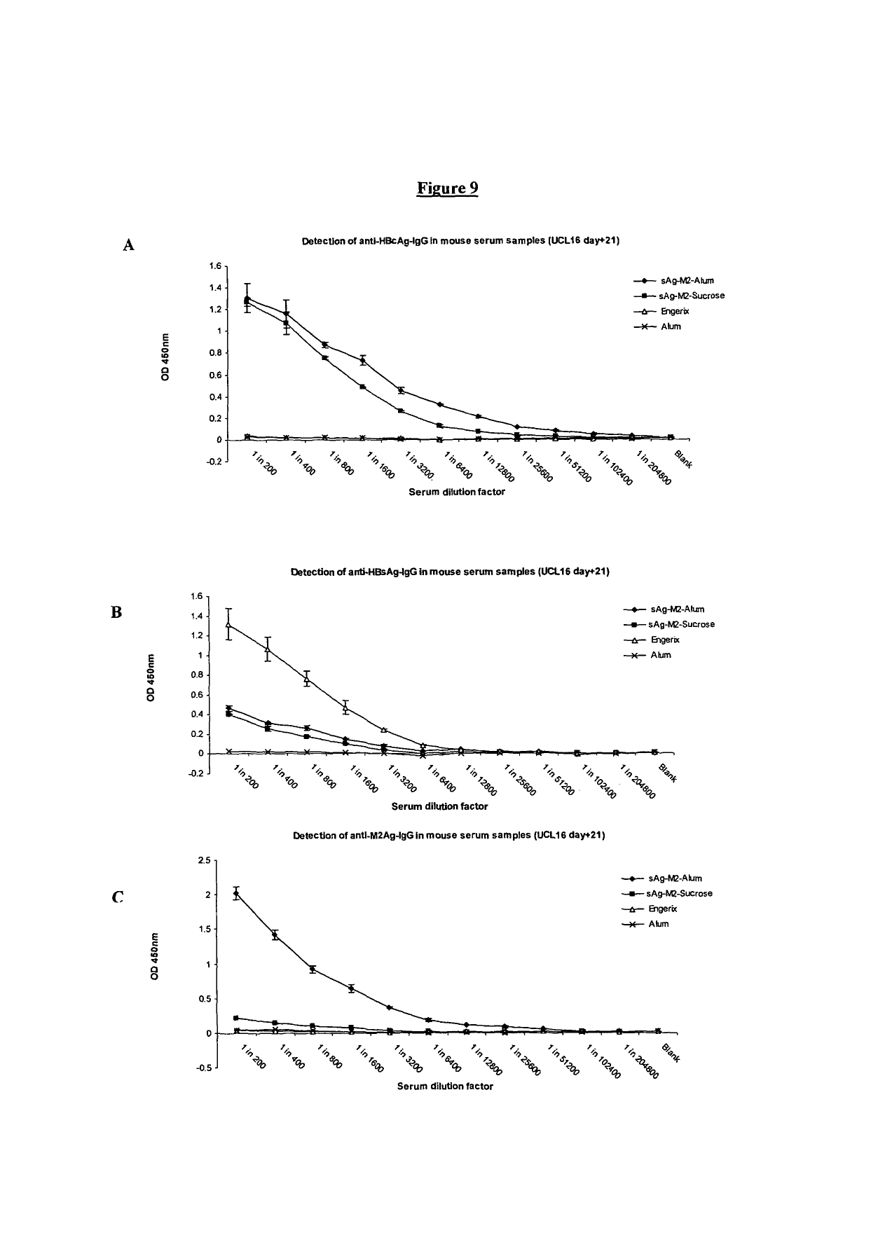

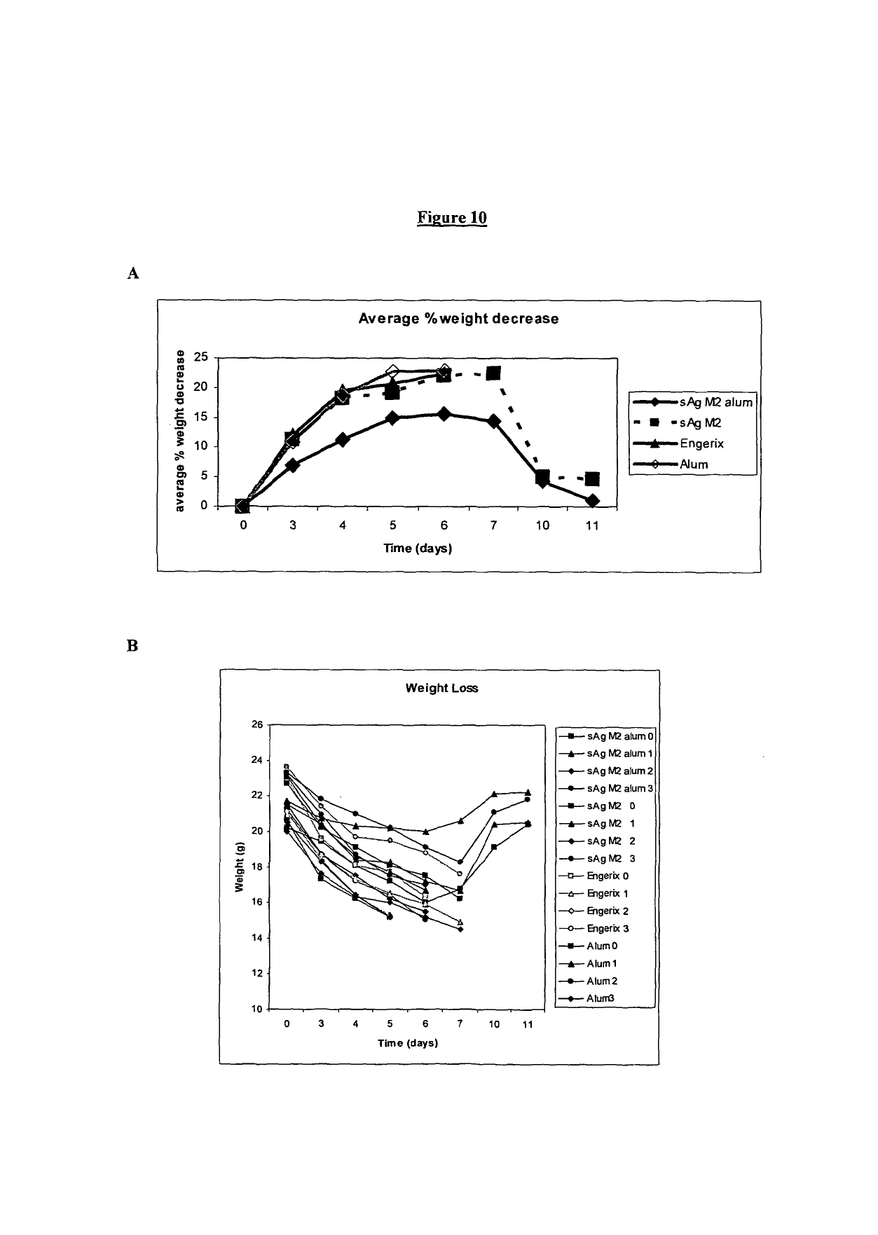

For sAg-M2, independent immune responses were generated to sAg, M2 and core protein, all initiated from a single protein vaccination (FIG. 9). The literature shows that the M2 protein itself is relatively immunologically silent. Therefore the data suggests that conjugation to HBV core markedly improves its immunogenicity. Furthermore, the M2 insert was found to confer protection to live influenza challenge. The percentage weight loss of mice vaccinated with sAg-M2 was significantly reduced and the overall survival of the mice significantly improved (FIG. 10).

The invention is therefore concerned with a novel influenza virus vaccine based on tandem hepatitis B (HBV) core proteins. In particular, the invention provides a protein comprising a first and a second copy of hepatitis B core antigen (HBcAg) in tandem, in which one or both of the copies of HBcAg comprises influenza virus A surface polypeptide M2 or a fragment thereof in the e1 loop.

### The Core Protein

The basic building block of the protein of the invention is HBcAg, which has 183 or 185 amino acids (aa) depending on the subtype of HBV. The sequence of the 183 amino acid protein of the ayw subtype plus a 29 amino acid pre-sequence is shown in SEQ ID NO: 2. The mature HBcAg runs from the Met residue at position 30 to the Cys residue at the extreme C-terminus, with the sequence from positions 1 to 29 being a pre-sequence.

The protein generally comprises only two copies of HBcAg forming a dimer because dimers of HBcAg form the structural building blocks of core particles. The HBcAg units are generally joined together in a head-to-toe fashion, i.e. the C-terminus of one unit is joined to the N-terminus of the adjacent unit. The units may be joined directly by a covalent bond (e.g. a peptide bond), but preferably they are joined by a linker which spaces the adjacent units apart and thereby prevents any problem with disruption of the packing of adjacent units. The nature of the linker is discussed below.

One or both of the HBcAg units in the protein of the invention may be native full length HBcAg. However, at least one of the units is a modified form of HBcAg having the influenza virus A surface polypeptide M2 or a fragment thereof inserted into the e1 loop. Both of the HBcAg units may have the influenza virus surface polypeptide M2 or a fragment thereof inserted into the e1 loop. When only one of the HBcAg units has the influenza virus surface polypeptide M2 or a fragment thereof inserted into the e1 loop, the other unit may be native HBcAg or it may have a heterologous epitope inserted into the e1 loop. Examples of possible heterologous epitopes are discussed below.

As a general rule, any modifications are chosen so as not to interfere with the conformation of HBcAg and its ability to assemble into particles. Such modifications are made at sites in the protein which are not important for maintenance of its conformation, for example in the e1 loop, the C-terminus and/or the N-terminus. The e1 loop of HBcAg can tolerate insertions of e.g. from 1 to 120 amino acids without destroying the particle-forming ability of the protein.

The HBcAg sequence may be modified by a substitution, insertion, deletion or extension. The size of insertion, deletion or extension may, for example, be from 1 to 200 aa, from 3 to 100 aa or from 6 to 50 aa. Substitutions may involve a number of amino acids up to, for example, 1, 2, 5, 10, 20 or 50 amino acids over the length of the HBcAg sequence. An extension may be at the N- or C-terminus of HBcAg. A deletion may be at the N-terminus, C-terminus or at an internal site of the protein. Substitutions may be made at any position in the protein sequence. Insertions may also be made at any point in the protein sequence, but are typically made in surface-exposed regions of the protein such as the e1 loop. An inserted sequence may carry a heterologous epitope. More than one modification may be made to each HBcAg unit. Thus, it is possible to make a terminal extension or deletion and also an internal insertion. For example, a truncation may be made at the C-terminus and an insertion may be made in the e1 loop. Each part of the HBcAg sequence in the protein of the invention preferably has at least 70% sequence identity to the corresponding sequence of a natural HBcAg protein, such as the protein having the sequence shown in SEQ ID NO: 2. More preferably, the identity is at least 80%, at least 90%, at least 98%, at least 97% or at least 99%. Methods of measuring protein homology are well known in the art and it will be understood by those of skill in the art that in the present context, homology is calculated on the basis of amino acid identity (sometimes referred to as “hard homology”).

For example the UWGCG Package (Devereux et al (1984) Nucleic Acids Research 12: 387-395) provides the BESTFIT program which can be used to calculate homology (for example used on its default settings). The PILEUP and BLAST algorithms can be used to calculate homology or line up sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased.

Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The e1 loop of HBcAg is at positions 68 to 90, and a heterologous epitope may be inserted anywhere between these positions. Preferably, the epitope is inserted in the region from positions 69 to 90, 71 to 90 or 75 to 85. Most preferred is to insert the epitope between amino acid residues 79 and 80 or between residues 80 and 81. When a heterologous epitope is inserted, the entire sequence of HBcAg may be maintained, or alternatively the whole or a part of the e1 loop sequence may be deleted and replaced by the heterologous sequence. Thus, amino acid residues 69 to 90, 71 to 90 or 75 to 85 may be replaced by a heterologous epitope. Where a heterologous epitope replaces el loop sequence, the epitope is generally not shorter than the sequence that it replaces.

A C-terminal truncation of HBcAg will generally not go beyond aa 144 because if any further truncation is made particles may not form. Thus, the deleted amino acids may, for example, comprise aa 144 to the C-terminal aa (aa 183 or 185), aa 150 to the C-terminal aa, aa 164 to the C-terminal aa or aa 172 to the C-terminal aa. The C-terminus of HBcAg binds DNA, and truncation of the C-terminus therefore reduces or completely removes DNA from preparations of HBcAg and HBcAg hybrid proteins.

The protein of the invention forms particles which preferably resemble the particles formed by native HBcAg. The particles of the invention are typically at least 10 nm in diameter, for example from 10 to 50 nm or from 20 to 40 nm in diameter, but preferably they are about 27 nm in diameter (which is the size of native HBcAg particles). They comprise multiple HBcAg units, for example from 150 to 300 units, but generally they are fixed to about 180 or about 240 units (which are the numbers of units in native HBcAg particles). As the protein of the invention is a dimer, this means that the number of protein monomers in the particles may be from 75 to 150 but is generally about 90 or about 120.

The linker between adjacent HBcAg units is generally a chain of amino acids at least 1.5 nm (15 Å) in length, for example from 1.5 to 10 nm, from 1.5 to 5 nm or from 1.5 to 3 nm. It may, for example, comprise 4 to 40 aa or 10 to 30 aa, preferably 15 to 21 aa. The linker is generally flexible. The amino acids in the linker may, for example, include or be entirely composed of glycine, serine and/or proline. A preferred linker comprises one or more repeats of the sequence GlyGlySer (GGS). Alternatively, the linker may comprise one or more GlyPro (GP) dipeptide repeats. The number of repeats may, for example, be from 1 to 18, preferably from 3 to 12. In the case of GGS repeats, the use of 5, 6 or 7 repeats has been found to allow the formation of particles. The linker may correspond to the hinge region of an antibody; this hinge region is thought to provide a flexible joint between the antigen-binding and tail domains of antibodies.

### Influenza Virus M2 Protein

The purpose of the protein of the invention is that it can be used to induce an immune response to influenza virus A and can therefore be used as an influenza virus A vaccine. The protein of the invention has the influenza virus A surface polypeptide M2 or a fragment thereof inserted into the e1 loop of one or both copies of HBcAg. A preferred protein of the invention has M2 or a fragment thereof inserted into the e1 loop of both copies of HBcAg.

The M2 protein is a proton-selective ion channel protein, integral in the viral envelope of the influenza virus. The channel itself is a homotetramer (consists of four identical M2 units), where the units are helices stabilized by two disulfide bonds. The M2 protein unit consists of three protein domains: the 24 amino acids on the N-terminal end, exposed to the outside environment, the 19 hydrophobic amino acids on the transmembrane region, and the 54 amino acids on the C-terminal end, oriented towards the inside of the viral particle. The full length sequence of M2 protein from influenza A virus strain A/34/PR8 is shown in SEQ ID NO: 3.

The M2 protein to be inserted into the e1 loop of HBcAg is derived from influenza virus A. It is preferably derived from the sequence in SEQ ID NO: 3. A full length M2 protein, i.e. the full 97 amino acid sequence, may be inserted into the e1 loop of HBcAg. For example, the full length sequence of SEQ ID NO: 3 may be inserted. The M2 protein may be a naturally occurring M2 protein or may be a variant of a naturally occurring M2 protein.

The sequence of the M2 protein may have homology with SEQ ID NO: 3, such as at least 60% identity, at least 80%, at least 90%, at least 95%, at least 97% or at least 99% identity, for example over the full sequence or over a region of at least 20, preferably at least 30, for instance at least 40, at least 50, at least 60, or at least 80 or more contiguous amino acids. Methods of measuring protein homology are well known in the art and are discussed above in relation to the HBV core protein.

The homologous protein typically differs from the naturally occurring M2 sequence by substitution, insertion or deletion, for example from 1, 2, 3, 4, 5 to 8 or more substitutions, deletions or insertions. The substitutions are preferably ‘conservative’ and may be made, for example, according to Table 1. Amino acids in the same block in the second column and preferably in the same line in the third column may be substituted for each other.

TABLE 1

| | | |
|---|---|---|
| ALIPHATIC | Non-Polar | G A P |
| | | I L V |
| | Polar—uncharged | C S T M |
| | | N Q |
| | Polar—charged | D E |
| | | K R |
| AROMATIC | | H F W Y |

A fragment of M2 protein to be used as an insert is a shortened version of a full length M2 protein that retains the ability of inducing an immune response. In some instances, a fragment may be at least 10%, such as at least 20%, at least 30%, at least 40% or at least 50%, preferably at least 60%, more preferably at least 70%, still more preferably at least 80%, even more preferably at least 90% and still more preferably at least 95% of the length of a naturally occurring M2 sequence such as the sequence of SEQ ID NO: 3. For example a fragment may be from 6 to 96 aa, from 6 to 50 aa or from 6 to 25 aa in length.

Preferably the fragment is from a region of M2 that is exposed on the surface of the virion. In a preferred embodiment of the invention the fragment is M2e, which is the external domain of the M2 protein. The M2e sequence from influenza A virus strain A/34/PR8 is shown as an insert in the sequence of FIG. 2 and is the first 24 amino acids of SEQ ID NO: 3). Preferably a fragment of M2 comprises amino acids 1 to 24 of SEQ ID NO: 3.

### Other Heterologous Epitopes

The flexibility of the tandem core system means that the protein of the invention, in addition to comprising M2 or a fragment thereof, may comprise one or more further heterologous epitopes. For example, the protein of the invention may comprise one or more further influenza virus epitopes in order to induce an excellent immune response to influenza virus. Alternatively, the protein of the invention may comprise one or more heterologous epitopes derived from a different source such as a different pathogen or allergen, in order to simultaneously induce immune responses to influenza virus and to a different pathogen or allergen.

Therefore, although the protein of the invention must have the influenza virus A surface polypeptide M2 or a fragment thereof inserted into the e1 loop of at least one copy of HBcAg, the e1 loop of the other copy of HBcAg in the protein may comprise any other type of heterologous epitope.

A “heterologous” epitope is an epitope that is not normally located at the position at which it is located in the HBcAg; it is generally from a protein other than HBcAg but it may be from a different location in HBcAg. For example, it may be HBV surface antigen (sAg). The epitope comprises a sequence of amino acids which raises an immune response. The epitope may be conformational or linear. It may be, for example, in a sequence of from 6 to 120 aa, from 6 to 50 aa or from 6 to 20 aa. A major advantage of the invention is that it allows epitopes carried on large sequences to be inserted into the e1 loop, for example on sequences of from 30 to 120 aa, 40 to 120 an or 60 to 120 aa.

The inventors have shown that it is possible to insert large protein sequences into the e1 loop of HBcAg in tandem constructs resulting in virus-like particle (VLP) formation. Even the relatively large GFP (27 kDa) insert did not disrupt VLP formation. The heterologous epitope to be used as an insert may therefore be of any suitable size that does not disrupt VLP formation. It is preferably less than 100 kDa, for example less than 80 kDa, less than 60 kDa, less than 40 kDa, less than 20 kDa, less than 10 kDa or less than 5 kDa.

The protein of the invention may contain more than one heterologous epitope, for example up to 2, 3, 5 or 8 heterologous epitopes, and in this case the epitopes may be present in the same or different HBcAg units. More than one copy of an epitope may be inserted in each HBcAg unit; for example, from 2 to 8 copies may be inserted. Where there are two or more heterologous epitopes in the protein of the invention, they may be from the same or different organisms and from the same or different proteins.

The epitope may be a T-cell or a B-cell epitope. If it is a T-cell epitope, it may be a cytotoxic T-lymphocyte (CTL) epitope or a T-helper (Th) cell epitope (e.g. a Th1 or Th2 epitope). In a preferred embodiment of the invention, one of the epitopes is a T-helper cell epitope and another is a B-cell or a CTL epitope. The presence of the T-helper cell epitope enhances the immune response against the B-cell or CTL epitope.

The choice of epitope depends on the disease that it is wished to vaccinate against. The epitope may, for example, be from a pathogenic organism, a cancer-associated antigen or an allergen. The pathogenic organism may, for example, be a virus, a bacterium or a protozoan.

The epitope may be derived from any pathogen, such as but not limited to, a virus, including a member of the orthomyxoviridae (including for instance influenza A, B and C viruses), adenoviridae (including for instance a human adenovirus), Caliciviridae (such as Norwalk virus group), herpesviridae (including for instance HSV-1, HSV-2, EBV, CMV and VZV), papovaviridae (including for instance Human Papilloma Viruse—HPV), poxviridae (including for instance smallpox and vaccinia), parvoviridae (including for instance parvovirus B19), reoviridae (including for instance a rotavirus), coronaviridae (including for instance SARS), flaviviridae (including for instance yellow fever, West Nile virus, dengue, hepatitis C and tick-borne encephalitis), picornaviridae (including enteroviruses, polio, rhinovirus, and hepatitis A), togaviridae (including for instance rubella virus), filoviridae (including for instance Marburg and Ebola), paramyxoviridae (including, a parainfluenza virus, respiratory syncitial virus (RSV), mumps and measles), rhabdoviridae (including for instance rabies virus), bunyaviridae (including for instance Hanta virus), retroviridae (including for instance HIV and HTLV—Human T-cell Lymphoma virus) and hepadnaviridae (including for instance hepatitis B).

The epitope may be derived from bacteria, including M. tuberculosis, Chlamydia, Ngonorrhoeae, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pnumophila, Yersinia pestis, Streptococcus (types A and B), Pneumococcus, Meningococcus, Hemophilus influenza (type b), Toxoplama gondii, Complybacteriosis, Moraxella catarrhalis, Donovanosis, and Actinomycosis, fungal pathogens including Candidiasis and Aspergillosis, and parasitic pathogens including Taenia, Flukes, Roundworms, Flatworms, Amebiasis, Giardiasis, Cryptosporidium, Schitosoma, Pneumocystis carinii, Trichomoniasis and Trichinosis.

The epitope may be derived from a pathogen that infects through a) the respiratory tract, b) the genito-urinary system or c) the gastrointestinal tract. Examples of such pathogens include a) members of the adenoviridae, paramyxoviridae and poxviridae, rhinovirus, influenza, and Hanta virus, b) Ureaplasma urealyticum, Neisseria gonorrhoeae, Gardnerella vaginalis, Trichomonas vaginalis, Treponema pallidum, Chlamydia trachomatis, Haemophilus ducreyi, herpes simplex virus, HPV, HIV, Candida albicans, Treponema pallidum, and Calmatobacterium granulomatis, and c) Shigella, Salmonella, Vibrio Cholera, E. coli, Entamoeba histolytica, Campylobacter, Clostridium, Yersinia, rotavirus, norovirus, adenovirus, astrovirus, Roundworms, Flatworms, Giardiasis, and Cryptosporidium.

The epitope to be used in the invention may be derived from a cancer such as, but not limited to, cancer of the lung, pancreas, bowel, colon, breast, uterus, cervix, ovary, testes, prostate, melanoma, Kaposi's sarcoma, a lymphoma (e.g. EBV-induced B-cell lymphoma) and a leukaemia. Specific examples of tumour associated antigens include, but are not limited to, cancer-testes antigens such as members of the MAGE family (MAGE 1, 2, 3 etc), NY-ESO-1 and SSX-2, differentation antigens such as tyrosinase, gp100, PSA, Her-2 and CEA, mutated self antigens and viral tumour antigens such as E6 and/or E7 from oncogenic HPV types. Further examples of particular tumour antigens include MART-1, Melan-A, p97, beta-HCG, GaINAc, MAGE-1, MAGE-2, MAGE-4, MAGE-12, MUC1, MUC2, MUC3, MUC4, MUC18, CEA, DDC, P1A, EpCam, melanoma antigen gp75, Hiker 8, high molecular weight melanoma antigen, K19, Tyr1, Tyr2, members of the pMel 17 gene family, c-Met, PSM (prostate mucin antigen), PSMA (prostate specific membrane antigen), prostate secretary protein, alpha-fetoprotein, CA125, CA19.9, TAG-72, BRCA-1 and BRCA-2 antigen.

Examples of other candidate epitopes for use in the invention include epitopes from the following antigens: the HIV antigens gp 120, gp 160, gag, pol, Nef, Tat and Ref; the malaria antigens CS protein and Sporozoite surface protein 2; the herpes virus antigens EBV gp340, EBV gp85, HSV gB, HSV gD, HSV gH, HSV early protein product, cytomegalovirus gB, cytomegalovirus gH, and IE protein gP72; the human papilloma virus antigens E4, E6 and E7; the respiratory syncytial virus antigens F protein, G protein, and N protein; the pertactin antigen of B. pertussis; the tumor antigens carcinoma CEA, carcinoma associated mucin, carcinoma P53, melanoma MPG, melanoma P97, MAGE antigen, carcinoma Neu oncogene product, prostate specific antigen (PSA), prostate associated antigen, ras protein, and myc; and house dust mite allergen.

Preferably, the heterologous epitope is derived from influenza virus. Therefore, when the protein of the invention is a dimer, one copy of HBcAg has M2 or a fragment thereof inserted into the e1 loop and the other copy of HBcAg may have a different influenza virus epitope inserted into the e1 loop. The different influenza virus epitope may be derived from influenza virus A, B or C. Preferably it is derived from influenza virus A. The heterologous epitope may be derived from any influenza antigens such as the HA (hemagglutinin), NA (neuraminidase), NP (nucleoprotein/nucleocapsid protein), M1, M2, PB1, PB2, PA, NS1 and NS2 antigens and in particular the HA, NA and M2 antigens.

### Making the Proteins of the Invention

The proteins of the invention are generally made by recombinant DNA technology. The invention includes a nucleic acid molecule (e.g. DNA or RNA) encoding a protein of the invention, such as an expression vector. The nucleic acid molecules may be made using known techniques for manipulating nucleic acids. Typically, two separate DNA constructs encoding two HBcAg units are made and then joined together by overlapping PCR.

A protein of the invention may be produced by culturing a host cell containing a nucleic molecule encoding the protein under conditions in which the protein is expressed, and recovering the protein. Suitable host cells include bacteria such as E. coli, yeast, mammalian cells and other eukaryotic cells, for example insect Sf9 cells.

The vectors constituting nucleic acid molecules according to the invention may be, for example, plasmid or virus vectors. They may contain an origin of replication, a promoter for the expression of the sequence encoding the protein, a regulator of the promoter such as an enhancer, a transcription stop signal, a translation start signal and/or a translation stop signal. The vectors may also contain one or more selectable marker genes, for example an ampicillin resistance gene in the case of a bacterial plasmid or a neomycin resistance gene in the case of a mammalian vector. Vectors may be used in vitro, for example for the production of RNA or used to transform or transfect a host cell. The vector may also be adapted to be used in vivo, for example in a method of gene therapy or DNA vaccination.

Promoters, enhancers and other expression regulation signals may be selected to be compatible with the host cell for which the expression vector is designed. For example, prokaryotic promoters may be used, in particular those suitable for use in E. coli strains (such as E. coli HB101). A promoter whose activity is induced in response to a change in the surrounding environment, such as anaerobic conditions, may be used. Preferably an htrA or nirB promoter may be used. These promoters may be used in particular to express the protein in an attenuated bacterium, for example for use as a vaccine. When expression of the protein of the invention is carried out in mammalian cells, either in vitro or in vivo, mammalian promoters may be used. Tissue-specific promoters, for example hepatocyte cell-specific promoters, may also be used. Viral promoters may also be used, for example the Moloney murine leukaemia virus long terminal repeat (MMLV LTR), the rous sarcoma virus (RSV) LTR promoter, the SV40 promoter, the human cytomegalovirus (CMV) IE promoter, herpes simplex virus promoters and adenovirus promoters. All these promoters are readily available in the art.

A protein according to the invention may be purified using conventional techniques for purifying proteins. The protein may, for example, be provided in purified, pure or isolated form. For use in a vaccine, the protein must generally be provided at a high level of purity, for example at a level at which it constitutes more than 80%, more than 90%, more than 95% or more than 98% of the protein in the preparation. However, it may be desirable to mix the protein with other proteins in the final vaccine formulation.

### Vaccination Against Diseases

The primary use of the protein of the invention is as a vaccine, particularly against influenza virus A. The invention includes a pharmaceutical composition (e.g. a vaccine composition) comprising a protein of the invention, a particle comprising multiple copies of the protein of the invention or a nucleic acid molecule encoding the protein of the invention and a pharmaceutically acceptable carrier or diluent. The composition can be used for vaccination of the human or animal body against influenza virus A.

The principle behind vaccination is to induce an immune response in a host so as to generate an immunological memory in the host. This means that, when the host is exposed to the virulent pathogen, it mounts an effective (protective) immune response, i.e. an immune response which inactivates and/or kills the pathogen. The invention forms the basis of a vaccine against influenza virus and depending on what other heterologous epitopes are included in the protein, could simultaneously vaccinate an individual to any of a wide range of other diseases and conditions, such as HBV, HAV, HCV, foot-and-mouth disease, polio, herpes, rabies, AIDS, dengue fever, yellow fever, malaria, tuberculosis, whooping cough, typhoid, food poisoning, diarrhoea, meningitis and gonorrhoea. The epitopes in the protein of the invention are chosen so as to be appropriate for the disease against which the vaccine is intended to provide protection.

The invention provides a method of inducing an immune response in a subject comprising administering to the subject the protein, particle or nucleic acid of the invention. Preferably the immune response is against influenza virus A.

The terms “individual” and “subject” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs as well as pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

In some instances, the invention may be administered to any suitable subject and in particular any suitable subject of a given species, preferably a suitable human subject. Thus, as many subjects as possible may, for instance, be subject to administration without emphasis on any particular group of subjects. For instance, a population of subjects as a whole, or as many as possible, may be subject to administration.

The protein, particle or nucleic acid of the invention is for administration to a subject. It may be administered simultaneously or sequentially with an adjuvant. Therefore the composition of the invention comprising the protein, particle or nucleic acid may also comprise an adjuvant. The composition of the invention may be one which is to be delivered by injection (such as intradermal, subcutaneous, intramuscular, intravenous, intraosseous, and intraperitoneal), transdermal particle delivery, inhalation, topically, orally or transmucosally (such as nasal, sublingual, vaginal or rectal).

The compositions may be formulated as conventional pharmaceutical preparations. This can be done using standard pharmaceutical formulation chemistries and methodologies, which are available to those skilled in the art. For example, compositions containing the protein, particle or nucleic acid with or without an adjuvant can be combined with one or more pharmaceutically acceptable excipients or vehicles to provide a liquid preparation. Thus also provided is a pharmaceutical composition comprising the protein, particle or nucleic acid together with a pharmaceutically acceptable carrier or diluent. The composition optionally comprises an adjuvant.

Auxiliary substances, such as wetting or emulsifying agents, pH buffering substances and the like, may be present. These carriers, diluents and auxiliary substances are generally pharmaceutical agents which may be administered without undue toxicity and which, in the case of antigenic compositions will not in themselves induce an immune response in the individual receiving the composition. Pharmaceutically acceptable carriers include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable carrier that serves as a stabilizer, particularly for peptide, protein or other like molecules if they are to be included in the composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

Certain facilitators of nucleic acid uptake and/or expression (“transfection facilitating agents”) can also be included in the compositions, for example, facilitators such as bupivacaine, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin™, and DOTAP (1,2-bis(oleyloxy)-3-(trimethylammonio)propane), see, e.g., Feigner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publication No. WO 93/19768).

Alternatively, the protein, particle or nucleic acid and/or the adjuvant may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules. For example, polynucleotides can be precipitated onto carriers in the presence of a polynucleotide condensing agent and a metal ion chelating agent. Preferred condensing agents include cationic polymers, in particular polyamines, and in particular a polyargine or a polylysine. In a preferred instance the polyamine is (Arg)4 or (Arg)6. Reference may be made to the techniques discussed in WO2004/208560 which may be employed. Once formulated the compositions can be delivered to a subject in vivo using a variety of known routes and techniques. For example, the liquid preparations can be provided as an injectable solution, suspension or emulsion and administered via parenteral, subcutaneous, intradermal, intramuscular, intravenous intraosseous and intraperitoneal injection using a conventional needle and syringe, or using a liquid jet injection system. Liquid preparations can also be administered topically to skin or mucosal tissue (e.g. nasal, sublingual, vaginal or rectal), or provided as a finely divided spray suitable for respiratory or pulmonary administration. Other modes of administration include oral administration, suppositories, and active or passive transdermal delivery techniques.

The protein, particle or nucleic acid of the invention is administered to a subject in an amount that will be effective in modulating an immune response. An appropriate effective amount will fall in a relatively broad range but can be readily determined by one of skill in the art by routine trials. The “Physicians Desk Reference” and “Goodman and Gilman's The Pharmacological Basis of Therapeutics” are useful for the purpose of determining the amount needed. Typically, the protein or particles are administered in a dose of from 0.1 to 200 mg, preferably from 1 to 100 mg, more preferably from 10 to 50 mg body weight. The nucleic acid of the invention may be administered directly as a naked nucleic acid construct using techniques known in the art or using vectors known in the art. The amount of nucleic acid administered is typically in the range of from 1 μg to 10 mg, preferably from 100 mg to 1 mg. The vaccine may be given in a single dose schedule or a multiple dose schedule, for example in from 2 to 32 or from 4 to 16 doses. The routes of administration and doses given above are intended only as a guide, and the route and dose may ultimately be at the discretion of the physician.

In some cases after an initial administration a subsequent administration of the composition of the invention may be performed. In particular, following an initial administration a subject may be given a “booster”. The booster may be, for instance, a dose chosen from any of those mentioned herein. The booster administration may, for instance, be at least a week, two weeks, four weeks, six weeks, a month, two months or six months after the initial administration.

The protein, particle or nucleic acid of the invention and an adjuvant may be administered sequentially or simultaneously, preferably simultaneously. The two entities may be administered in the same or different compositions, preferably the same composition. An adjuvant is delivered so that an adjuvant effect is seen, that is the immune response seen will differ from that if the adjuvant had not been administered with the antigen. The two entities may be administered at the same or different sites, preferably the same sites. Preferably, the two entities are administered in the same composition at the same site at the same time preferably via injection.

Any suitable adjuvant may be used. Currently used vaccine adjuvants include:

Inorganic compounds, such as aluminium salts (e.g. aluminium hydroxide and aluminium phosphate) or calcium phosphate. Aluminium salts are otherwise known as alum.

Oil emulsions and surfactant based formulations, e.g. MF59 (microfluidised detergent stabilised oil-in-water emulsion), QS21 (purified saponin), AS02 [SBAS2] (oil-in-water emulsion+MPL+QS-21), Montanide ISA-51 and ISA-720 (stabilised water-in-oil emulsion).

Particulate adjuvants, e.g. virosomes (unilamellar liposomal vehicles incorporating e.g. influenza haemagglutinin), ASO4 ([SBAS4] Al salt with MPL), ISCOMS (structured complex of saponins and lipids), and polylactide co-glycolide (PLG).

Microbial derivatives (natural and synthetic), e.g. monophosphoryl lipid A (MPL), Detox (MPL+M Phlei cell wall skeleton), AGP [RC-529] (synthetic acylated monosaccharide), DC_Chol (lipoidal immunostimulators able to self organise into liposomes), OM-174 (lipid A derivative), CpG motifs (synthetic oligonucleotides containing immunostimulatory CpG motifs), and modified LT and CT (genetically modified bacterial toxins to provide non-toxic adjuvant effects).

Endogenous human immunomodulators, e.g. hGM-CSF or hIL-12 (cytokines that can be administered either as protein or plasmid encoded), and Immudaptin (C3d tandem array).

Inert vehicles, such as gold particles.

Preferably the adjuvant used is alum. Most preferably the adjuvant is a mixture of aluminium hydroxide and magnesium hydroxide, for example Inject alum (Pierce Laboratories).

The invention is illustrated by the following Example:

### EXAMPLE

Two different tandem core constructs were designed, both with dual inserts. The first contained HBV surface antigen (sAg) in core 1 and green fluorescent protein (GFP) in core 2. The second comprised sAg in core 1 but had the M2 protein from influenza in core 2. These were expressed in bacteria, purified and then tested in vivo for immunogenicity.

### Design of Constructs

All tandem core clones are derived from the parental construct CoHo7e. Segments of tandem HBV core sequence were prepared using overlapping oligonucleotides and PCR technology and the resulting sequences assembled to form CoHo7e in the pET28b expression vector (Novagen).

CoHo7sAg-Empty Parent Construct.

Both CoHo7sAg,eGFPs and CoHo7sAg,M2e are derived from CoHo7sAg,e, a tandem core construct containing HBsAg encoding sequence in the first core el-loop with an empty e1 loop in the second core. This parental construct was prepared by insertion of the HBsAg sequence into the construct CoHo7e as follows:

HBsAg sequence encoding amino acids 108-155 (strain Adw) was PCR amplified using oligonucleotide primers that introduce Bam HI and Not I restriction enzyme sites to the 5′ and 3′ ends respectively. The resulting PCR product and CoHo7e plasmid DNA were each digested with the Barn HI and Not I and the appropriate vector and PCR product insert DNA were agarose gel purified. The vector and insert were ligated using T4 DNA ligase and the resulting ligated plasmids were used to transform E. coli DH5α. Colonies were PCR screened for the presence of the HBsAg insert sequence and positive clones grown for plasmid preparation and restriction digestion analysis.

CoHo7sAg,eGFPs Construct.

This construct is a homo-7-linker tandem with HBV surface antigen (residues 108-155, adw) in 1st core (el loop) and green fluorescent protein in the 2nd core (e1 loop). To generate this construct the green fluorescent protein (eGFP) sequence was initially inserted into CoHe7e to form CoHe7e,eGFPs as follows:

The eGFP sequence was amplified by PCR using oligonucleotide primers that introduce sequence encoding amino acids GGS (spacer sequence), Eco RI and Nhe I restriction enzyme sites to the 5′ and 3′ ends respectively. The resulting PCR product and CoHo7e plasmid DNA were each digested with the Eco RI and Nhe I and the appropriate vector and PCR product insert DNA were agarose gel purified. The vector and insert were ligated using T4 DNA ligase and the resulting ligated plasmids were used to transform E. coli DH5α. Colonies were PCR screened for the presence of the eGFP insert sequence and positive clones grown for plasmid preparation and restriction digestion analysis.

To obtain an eGFP insert for assembly of the CoHo7sAg,eGFP, the eGFP sequence was cut from CoHe7e,eGFP by restriction digestion with Eco RI and Nhe I. Likewise, the CoHo7sAg,e vector was digested with Eco RI and Nhe I. The vector and insert DNA were agarose gel purified and ligated using T4 DNA ligase. The resulting ligated plasmids were used to transform E. coli DH5α. Colonies were PCR screened for the presence of the eGFP insert sequence and positive clones grown for plasmid preparation and restriction digestion analysis.

CoHo7sAg,M2e Construct.

This construct is a homo-7-linker tandem with HBV surface antigen (residues 108-155, adw) in 1st core (e1 loop) and influenza M2 protein (residues 1-24) in the 2nd core (e1 loop).

The influenza M2e (matrix protein 2 ectodomain) sequence was taken from the published influenza A virus strain A/34/PR8 genomic sequence. Since this 72 nt sequence is spliced over the M1 gene, it was assembled by annealing complementary oligonucleotides encoding the M2e protein only. The resulting DNA was amplified by PCR using oligonucleotide primers that introduce Eco RI and Nhe I restriction enzyme sites to the 5′ and 3′ ends respectively. The resulting PCR product was directly ligated into cloning vector pJET (Fermentas). The M2e insert sequence was then excised from the resulting plasmid using restriction enzymes Eco RI and Nhe I. The CoHo7sAg,e vector was also digested with Eco RI and Nhe I and the vector and insert agarose gel purified. The vector and insert were ligated using T4 DNA ligase and the resulting ligated plasmids were used to transform E. coli DH5α. Colonies were PCR screened for the presence of the M2e insert sequence and positive clones grown for plasmid preparation and restriction digestion analysis.

### Purification of Protein

### Expression of Tandem Core Constructs

E. coli BL21(DE3) competent cells were transformed with tandem core vectors encoding coHo7sAg, eGFPs and coHo7sAg, M2e from which 400 ml cultures were grown from individual colonies in 2 litre baffled flasks. Protein expression was induced overnight at 16° C. with 1 mM IPTG once cultures were past mid-log phase, typically OD_{600 nm} of 0.8-1.0. Cells were harvested prior to resuspension in lysis buffer (10 mM Tris pH 8.5, 5% glycerol v/v, protease inhibitor cocktail (Sigma), 5 mM DTT). Cell disruption was performed by French press (2 passes at 16,000 psi) and the non-specific nuclease, Benzonase (Novagen), added after each pass. This lysate was further disrupted by sonication on ice (6 cycles of 10 seconds on, 20 seconds off) and 10 mM EDTA added. The tandem cores were further extracted from the lysate suspension by adding 0.05% Tween 20 and incubated 1 hour at room temperature. Soluble crude lysates were obtained by collecting the supernatant fluid after a clarification spin (1 hour, 26-50,000×g at 8° C.) to remove cell debris and other insoluble material.

sAg-GFP Construct

The coHo7sAg, eGFPs VLPs were enriched from the clarified lysate by sedimentation (3 hours, 150,000×g at 8° C.) through a single 30% sucrose cushion and the resultant pellet material soaked in buffer (10 mM Tris pH 8.5, 150 mM NaCl) prior to resuspension in a final volume of 5 ml and brief sonication. This VLP suspension was further purified by overlaying on a discontinuous sucrose gradient made up of 5 ml steps of 60% and 40% sucrose and topped with a 20 ml step of 30% sucrose. The resultant sedimented VLPs were collected in 1 ml fractions and identified by SDS-PAGE, western blot, ELISA and Bradford assay analyses.

9×1 ml fractions (#5−11 and #13+14) were pooled from a discontinuous sucrose gradient, diluted in to a final volume of 30 ml Tris/NaCl solution and sonicated. This solution was concentrated by sedimentation through 30% sucrose solution. The resulting pellet was soaked in 1 ml of 20 mM Tris pH 8, 150 mM NaCl buffer, then thoroughly mixed by pipetting and briefly sonicated prior to transferring to a clean 15 ml falcon tube. The immunogen was mixed with sucrose to give 40% solution, adjusting the total protein concentration to ˜0.285 mg/ml. The sample was stored at 4° C. prior to shipping.

sAg-M2 Construct

The coHo7sAg, M2e VLPs were enriched from the clarified lysate by sedimentation (3 hours, 150,000×g at 8° C.) through a two-step sucrose cushion (30% and 50% sucrose) and the resulting separated material collected in lml fractions. Fractions of interest were identified by SDS-PAGE, western blot, ELISA and Bradford assay analyses.

Fractions (#3-8) were pooled from a 2-step sucrose cushion (30/50%), dialysed overnight into Tris/NaCl/EDTA/DTT solution and sonicated. This solution was concentrated to ˜5 ml volume with a 50,000 MWCO Centriprep (Amicon) centrifugal filtration device. The suspension was briefly sonicated and filtered (0.45 μm) prior to gel filtration over a 16/26 Sephacryl S-500HR column, sample injection (2 ml) was repeated twice collecting eluted 2 ml fractions. Peak fractions, equivalent to the column void volume (#5), from the repeated runs were pooled and dialysed in 20 mM Tris pH 8, 150 mM NaCl buffer. The immunogen was diluted to a final concentration of ˜0.2 mg/ml in a buffer composition of 20 mM Tris pH 8, 150 mM NaCl and either mixed 1:1 with Imject (alum) adjuvant, or sucrose added to 40% (w/v). Samples were stored at 4° C. prior to shipping.

### Protein Identity

sAg-eGFP Construct
Immunogen sample: coHo7sAg,eGFP (69 kDa)
Initial appearance: Slightly opaque suspension free of any visible clumps or aggregates.
Buffer composition: 20 mM Tris pH 8.0
   150 mM NaCl
   40% sucrose (w/v)
Final Concentration: (Total protein) 0.285 mg/ml
Purity (estimate): ≧50%
See FIG. 6
   sAg-M2 Construct
Immunogen sample: coHo7sAg,M2e (44 kDa)
Initial appearance: (Imject) milky white suspension
   (Sucrose) clear suspension free of any visible clumps or aggregates.
Buffer composition: 20 mM Tris pH 8.0
   150 mM NaCl
   40% Sucrose or Imject alum
Final Concentration: 0.2 mg/ml (1:1 imject to immunogen)
   0.2 mg/ml (40% sucrose)
Purity (estimate): ≧50%

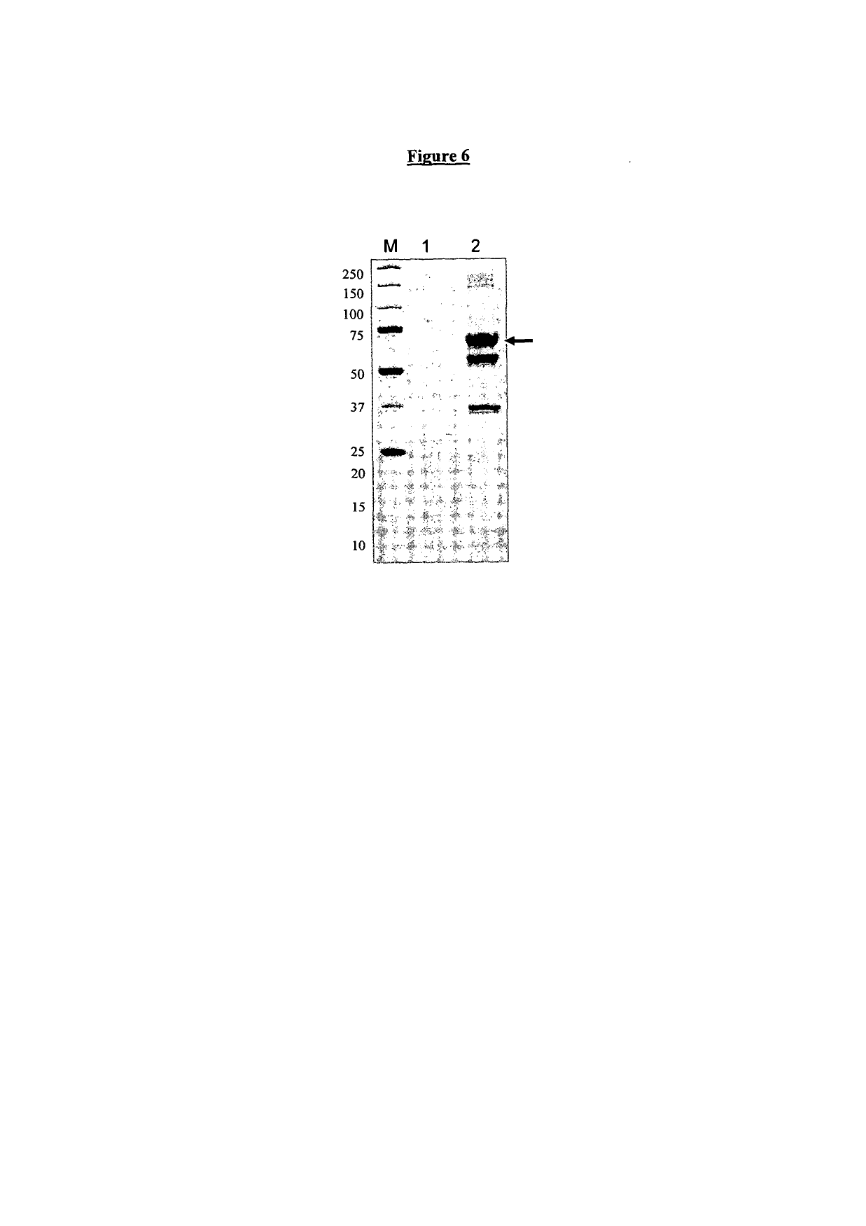

### See FIG. 7

### Methods

### In Vivo Testing

Groups of eight mice (6 wk old or greater Balb/c females (Charles River, UK)) were vaccinated with tandem-core constructs in a maximum volume of 200 μl. All dilutions were carried our in non-pyrogenic saline (Braun, Germany). Control animals received 200 μl of Engerix B (GSK, UK) or Imject alum (Pierce Laboratories). This was repeated on a weekly basis for three weeks. Animals were observed weekly and tail bled on a weekly basis after day 0.

Serum immune responses were examined to both inserts and the core protein. Specifically, ELISAs were carried out examining antibody responses to sAg, GFP, M2 peptide (MSLLTEVETPIRNEWGCRCNGSSD-OH) (the first 24 amino acids of SEQ ID NO: 3) or HBV core protein (see below).

In animals tested with sAg-M2 tandem core constructs, half of each group (×4 mice) were challenged with live influenza virus (strain PR8) once seropositivity had been confirmed. Animals were anaesthetised, challenged with 40 μl of virus (2500U; 20 μl per nostril) and then weight loss/clinical symptoms monitored daily. Sacrifice was compulsory if weight loss exceeded 25% of the initial weight or clinical score exceeded 10 points.

### ELISA Testing

M2 ELISA protocol: M2 peptide was diluted to a concentration of 6.25 μg/ml in ELISA coating buffer (PBS+0.6M NaCl) and incubated on the ELISA plate (Nunc, maxisorp) overnight at 4° C. The ELISA plate was blocked with 10% dried milk in PBS-T for 1 hour at 37° C. and the wells were washed in phosphate buffered saline plus 0.05% Tween-20 pH 7.4 (PBS-T) four times. The M2 peptide was detected by incubation with serum from M2 immunised mice or Polyclonal Rabbit anti-M2-IgG for 1 hour at 37° C. The wells were washed and then the secondary antibody Goat anti-mouse IgG-HRP (1 in 5000 dilution) or Goat anti-rabbit IgG-HRP (1 in 5000 dilution) was incubated for 1 hour at 37° C. Plates were washed and then detected by adding 100 ul TMB. The reaction was stopped by adding 50 μl 1M H₂SO₄. The OD was measured at 450 nm.

GFP ELISA protocol: Recombinant GFP was diluted to a concentration of 500 ng/ml in ELISA coating buffer (50 mM carbonate bicarbonate buffer) and incubated on the ELISA plate overnight at 4° C. The ELISA plate was blocked with 10% dried milk in PBS-T for 1 hour at 37° C. and the wells were washed in PBS-T four times. The GFP was detected by incubation with serum from GFP immunised mice for 1 hour at 37° C. The wells were washed and then the secondary antibody Goat anti-mouse IgG-HRP (1 in 5000 dilution) was incubated for 1 hour at 37° C. Plates were washed and then detected by adding 100 ul TMB. The reaction was stopped by adding 50 μl 1M H₂SO₄. The OD was measured at 450 nm.

HBc ELISA protocol: Recombinant HBc was diluted to a concentration of 500 ng/ml in ELISA coating buffer (50 mM carbonate bicarbonate buffer) and incubated on the ELISA plate overnight at 4° C. The ELISA plate was blocked with 10% dried milk in PBS-T for 1 hour at 37° C. and the wells were washed in PBS-T four times. HBc was detected by incubation with serum from HBc immunised mice for 1 hour at 37° C. The wells were washed and then the secondary antibody Goat anti-mouse IgG-HRP (1 in 5000 dilution) was incubated for 1 hour at 37° C. Plates were washed and then detected by adding 100u1 TMB. The reaction was stopped by adding 50 μl 1M H₂SO₄. The OD was measured at 450 nm.

HBs ELISA protocol: Recombinant HBs was diluted to a concentration of 500 ng/ml in ELISA coating buffer (50 mM carbonate bicarbonate buffer) and incubated on the ELISA plate overnight at 4° C. The ELISA plate was blocked with 10% dried milk in PBS-T for 1 hour at 37° C. and the wells were washed in PBS-T four times. HBs were detected by incubation with serum from HBs immunised mice or serum form Engerix B immunised mice for 1 hour at 37° C. The wells were washed and then the secondary antibody Goat anti-mouse IgG-HRP (1 in 5000 dilution) was incubated for 1 hour at 37° C. Plates were washed and then detected by adding 100 ul TMB. The reaction was stopped by adding 50 μl 1M H2SO₄. The OD was measured at 450 nm.

### Results

Antibody Response to sAg-GFP

In order to examine the nature of the antibody response to tandem core constructs expressing inserts, a complete series expressing one or both antigens was made. In this series of experiments the constructs were (i) sAg-empty (ii) empty-GFP and (iii) sAg-GFP. In all cases, electron microscopy confirmed that VLPs readily formed, although interestingly the best morphology was seen in the empty-GFP construct.

ELISA data prepared using sera taken at day +21 are shown in FIG. 8 however it should be noted that responses were detectable as soon as day +7 after the last vaccination. All three constructs, as detailed above, contain core protein and hence it was reasonable to assume that all should give a strong anti-core response. This was largely true with the notable exception of empty-GFP. One possible reason for this may be that the exceptionally well formed morphology of this VLP was such that folding was so precise that the GFP insert completely covered the surface of the macromolecule thus making the core protein inaccessible to antibody.

Antibody responses to GFP were much more predictable and strong responses were seen for both single (empty-GFP) and dual insert (sAg-GFP) constructs. However, surface antigen antibody responses were not as expected. The positive control of

Engerix B validated assay conditions however only the single sAg-empty construct showed any detectable HBV response. The most likely explanation for this is that the large GFP (27kDa) insert sterically hinders the availability of the much smaller sAg (4.8kDa) insert. Thus, although these data clearly demonstrate that large inserts can indeed be accommodated by tandem core, care must be taken as to the orientation of the antigenic insert.

Antibody Response to sAg-M2

Further investigation into the interactions between different inserts in each core was carried out by designing a second construct containing sAg in core 1 and M2 (influenza) in core 2. In this case, both inserts are relatively small (4.8 and 2.9 kDa respectively). Mice were immunised with this construct either with or without alum as an adjuvant.

Core responses were strong at day 21 irrespective of the presence of adjuvant (FIG. 9). Similarly, sAg responses were strong in both adjuvant and sucrose buffers. Whilst levels were not equivalent to Engerix B, they were considerably stronger than those seen previously. Furthermore, it may be that the protein concentration of Engerix is not directly comparable to tandem core and a future titration study will be designed to address this. Responses to the second insert were also strong, although there was a notable difference between animals vaccinated with sAg-M2 on alum compared with the same construct formulated in sucrose buffer.

### Influenza Protection

The strength of the antibody response to M2 was of such magnitude that a challenge experiment was carried out in which a fatal dose of influenza virus strain PR8 was administered to each group after vaccination. Previous data have established that a dose of 2500U virus ensured 100% infection and that weight loss and clinical scores make excellent surrogates for fatal endpoints. UK Home Office animal licensing procedures have deemed that a weight loss of greater than 25% may be considered unrecoverable and animals must be sacrificed if this occurs. FIG. 10 shows the percentage weight loss for each group and also the absolute weights of each individual animal. It is clear that animals vaccinated with sAg-M2 formulated on alum showed lower average weight loss and indeed recovered after six days. This was not true for any of the other groups, although a small but detectable improvement in weight was also seen in the sAg-M2 group presented on sucrose. It should be noted, however, that even in the group that recovered, a clinical infection was still manifest.

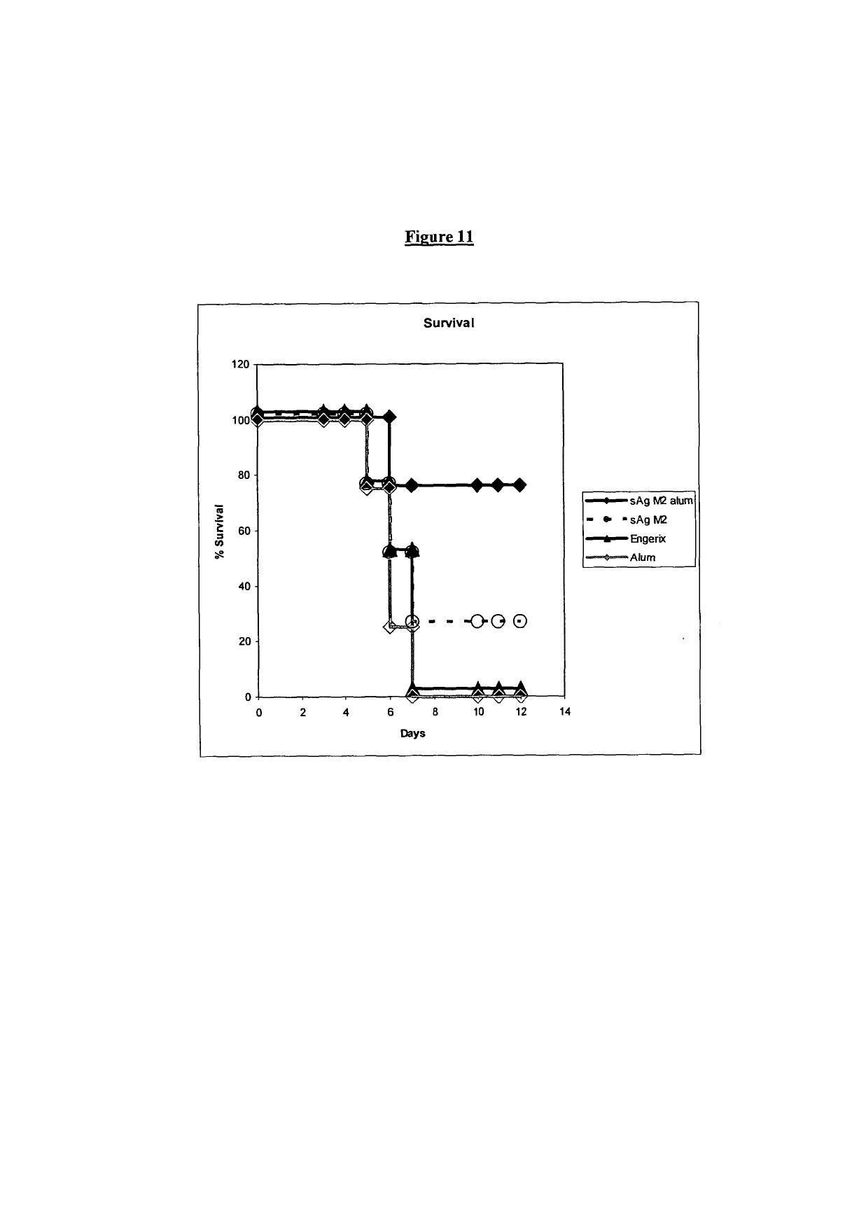

This result is confirmed when overall survival is recorded (FIG. 11). Animals vaccinated with sAg-M2 on alum showed 75% survival, whilst sAg-M2 on sucrose gave 25%. Both Engerix B and alum controls showed no protection and had similar end-points at seven days post-infection.

### Discussion

The immunogenicity of core protein is well established, as is its ability to accept antigenic inserts into its MIR. However, it is equally well documented that the technology has a major weakness because the core dimers no longer form when large or hydrophobic inserts are added, leading to a failure of VLP formation. The development of tandem core constructs overcomes this major limitation. The data presented in this Example clearly illustrate two major concepts; (1) large inserts can be accommodated by tandem core constructs resulting in VLP formation (2) constructs expressing dual inserts do lead to two different immune responses.

The inventors have shown that monomeric and tandem core VLPs appear identical (FIG. 5). Furthermore, all constructs used in these studies contained VLPs, as confirmed by electron microscopy. Therefore, even the presence of the relatively large GFP (27 kDa) insert did not disrupt this formation. It is notable that in the case of empty-GFP, the folding may even have been superior to that of unloaded tandem cores, as evidenced by the failure to detect an anti-core response in this construct (FIG. 8). The most likely explanation for this is that the GFP molecules decorated the surface of the resulting VLP, thereby masking the core protein itself, and giving rise to a very stable and tightly packed macromolecule.

However, these data also suggest that their may be interaction between inserts placed in adjacent cores. The lack of a sAg response when sAg-GFP is used (FIG. 8) suggests that the larger GFP molecule may be sterically hindering the availability of the smaller sAg antigen to antibody. The detection of sAg responses after immunisation with sAg-empty confirms the presence of the relevant epitope and so therefore it is likely that the interaction between antigenic inserts is the reason for the lack of sAg response when a dual insert is employed. Currently, genetic modification of the tandem core construct is being carried out to try and dissociate the insert interactions between adjacent MIRs.

A second construct was then produced using sAg and M2 in order to prove that dual inserts could indeed generate two independent immune responses to inserts from a single recombinant protein. FIG. 9 proves conclusively that this is the case, with responses to sAg, M2 and core protein all initiated from a single protein vaccination. Indeed, it is notable that literature shows that the M2 protein itself is relatively immunologically silent and so these data suggest that conjugation of M2 to core also markedly improves its immunogenicity.

Furthermore, the M2 insert confers protection to live influenza challenge, however, animals did manifest clinical symptoms, albeit at reduced levels. This suggests that whilst the vaccine is effective in reducing influenza infection, it does not produce full sterile immunity. Hence, improvements could be made by adding a second influenza antigen. The nature of tandem core constructs is such that it is an ideal vehicle for the addition of a second immunogenic epitope, leading to the generation of even stronger immune responses.

Tandem core has thus shown an ability to generate a novel influenza vaccine, but its utility is not limited to this alone. Indeed, these data clearly show that two disparate immune responses can be generated simultaneously and so this could be harnessed by making a vaccine against two different infections concurrently or by improving protection rates by using two antigens from the same target together. These data are likely to be applicable to a number of viral and cancerous diseases using disease specific inserts.

1. A protein comprising a first and a second copy of hepatitis B core antigen (HBcAg) in tandem, in which one or both of the copies of HBcAg comprises influenza virus A surface polypeptide M2 or a fragment thereof in the e1 loop.

2. The protein according to claim 1, wherein the first copy of HBcAg comprises said influenza virus A surface polypeptide M2 or fragment thereof in the el loop and the second copy of HBcAg comprises another heterologous epitope in the e1 loop.

3. The protein according to claim 2, wherein the second heterologous epitope is from an influenza virus antigen.

4. The protein according to claim 3, wherein the second heterologous epitope is from HA, NA or Ml.

5. The protein according to claim 1, wherein both copies of HBcAg comprise the influenza virus A surface polypeptide M2 or a fragment thereof in the el loop.

6. The protein according to claim 1, wherein the fragment of influenza virus A surface polypeptide M2 is the M2 ectodomain (M2e).

7. The protein according to claim 1, wherein one or more of the copies of HBcAg is truncated at the C-terminus.

8. The protein according to claim 1, wherein the tandem copies of HBcAg are joined by a linker.

9. The protein according to claim 8, wherein the linker is at least 1.5 nm in length.

10. The protein according to claim 8, wherein the linker comprises multiple copies of the sequence GlyGlySer (GGS).

11. A particle comprising multiple copies of a protein according to claim 1.

12. A nucleic acid molecule encoding a protein according to claim 1.

13. The nucleic acid molecule according to claim 12, which is an expression vector.

14. A host cell comprising a nucleic acid molecule according to claim 12.

15. A process for producing a protein according to claim 1, which process comprises culturing a host cell containing a nucleic acid molecule which encodes the protein under conditions in which the protein is expressed, and recovering the protein.

16. A pharmaceutical composition comprising:
a protein comprising a first and a second copy of hepatitis B core antigen (HBcAg) in tandem, in which one or both of the copies of HBcAg comprises influenza virus A surface polypeptide M2 or a fragment thereof in the e1 loop;
a particle comprising multiple copies of a protein comprising a first and a second copy of hepatitis B core antigen (HBcAg) tandem, in which one or both of the copies of HBcAg comprises influenza virus A surface polypeptide M2 or a fragment thereof in the e1 loop; or
a nucleic acid molecule encoding a protein comprising a first and a second copy of hepatitis B core antigen (HBcAg) in tandem, in which one or both of the copies of HBcAg comprises influenza virus A surface polypeptide M2 or a fragment thereof in the e1 loop.

17. The pharmaceutical composition of claim 16, further comprising an adjuvant.

18.-21. (canceled)

22. A method of inducing an immune response in a subject, which method comprises administering to the subject a protein comprising a first and a second copy of hepatitis B core antigen (HBcAg) in tandem, in which one or both of the copies of HBcAg comprises influenza virus A surface polypeptide M2 or a fragment thereof in the e1 loop;
a particle comprising multiple copies of a protein comprising a first and a second copy of hepatitis B core antigen (HBcAg) in tandem, in which one or both of the copies of HBcAg comprises influenza virus A surface polypeptide M2 or a fragment thereof in the e1 loop; or
a nucleic acid molecule encoding a protein comprising a first and a second copy of hepatitis B core antigen (HBcAg) in tandem, in which one or both of the copies of HBcAg comprises influenza virus A surface polypeptide M2 or a fragment thereof in the e1 loop.

23. The method according to claim 22, for inducing an immune response against influenza.

24. The method according to claim 22, wherein administration is in combination with an adjuvant.
